(19) United States
(12) Patent Application Publication
(10) Pub. No.: US 2012/0258076 A1
(43) Pub. Date: Oct. 11, 2012

(54) METHOD FOR CHARACTERIZING HOST IMMUNE FUNTION BY EX VIVO INDUCTION OF OFFENSIVE AND DEFENSIVE IMMUNE MARKERS

(75) Inventor: Masato Mitsuhashi, Irvine, CA (US)

(73) Assignees: HITACHI CHEMICAL RESEARCH CENTER, INC., Irvine, CA (US); HITACHI CHEMICAL CO., LTD., Tokyo (JP)

(21) Appl. No.: 13/516,552

(22) PCT Filed: Dec. 8, 2010
(86) PCT No.: PCT/US10/59552

§ 371 Date: Jun. 15, 2012

Related U.S. Application Data
(60) Provisional application No. 61/287,114, filed on Dec. 16, 2009.

(51) Int. Cl. C12Q 1/68; A61K 38/20; A61K 31/716

(52) U.S. Cl. 424/85.2; 435/6.11; 514/54

(57) ABSTRACT

A host's immune function can be characterized by quantifying changes in offensive and defensive immune function associated markers. Certain methods can be used to identify a potentially efficacious therapy for a subject based on the induction of expression of offensive and defensive immune function-associated markers. Additionally, some methods can be used to identify drugs that allow the stimulation of either the offensive or defensive immune response while inhibiting the other of offensive or defensive immune response.

### CROSS-REFERENCE TO RELATED APPLICATIONS

This application claims the benefit of U.S. Provisional Application No. 61/287,114, filed on Dec. 16, 2009, the disclosure of which is expressly incorporated by reference herein.

### BACKGROUND

1. Field of the Invention

The present disclosure relates to markers that can readily be measured and are associated with either the offensive or defensive immune function of a host. More specifically, the present disclosure relates to the ex vivo induction of certain markers associated with either offensive (attacking foreign bodies) or defensive (regulating offensive immune activity) immune function and measurements of their induction as a predictor of host responsiveness to an infection and/or as a method for screening drugs for immuno-modulatory effects. Data generated with respect to gene induction can be used to identify therapies that are specifically tailored to the expression profile of a particular subject.

2. Description of Related Art

The immune system comprises a set of diverse proteins, cells, tissues, and processes that protect a host from disease by first identifying and then eliminating pathogens and tumor cells. A primary role of the immune system is to distinguish foreign cells or pathogens from endogenous cells, in other words, distinguishing between “self” and “non-self.” Cells that are endogenous to the host are thought to be recognized as “self” by the expression of Class I Major Histocompatibility Complex (MHC). Those cells without Class I MHC or with reduced levels of expression may be targeted by the immune system as damaged “self” or “non-self” cells. Despite this elegant system, disorders in the immune system can lead to disease, including immunodeficiency, carcinogenesis, or autoimmunity.

White blood cells (WBCs; leukocytes) are the primary functional class of cells in the immune system. While several subtypes of WBCs exist, lymphocytes are one subtype that play an integral role in the immune system defense mechanisms. Natural killer (NK) cells are a specialized type of cytotoxic lymphocyte that are involved in the identification and rejection of tumor cells, virally infected cells, or damaged “self” cells. Cytotoxic T cells are another lymphocyte sub-group that are capable of inducing the death of infected somatic or tumor cells. Once activated, often by cytokines or presentation of a foreign antigen, NK cells and cytotoxic T cells release small granules from their cytoplasm, which contain various proteins and proteases. Certain proteins, such as perforin, induce pore formation in the membrane of a targeted cell, allowing proteases, such as granzymes, to enter the cells and induce the programmed cell death process (apoptosis).

Concurrently with the attack on foreign cells or tumor cells, the immune system also initiates a negative feedback loop to limit the activity of the immune system to shut down the immune system after a successful elimination of foreign cells and also to avoid hyper-responsiveness and possible attacking of “self” cells or other pathways leading to development of auto-immunity. Regulatory T cells actively suppress activation of the immune system and the critical nature of this role is evidenced by the severe autoimmune syndrome that results from a genetic deficiency in regulatory T cells (T-reg). Myeloid-derived suppressor cells (MDSCs) are also involved in the immune down-regulating process, as the MDSCs block the binding of cytotoxic T cells to the foreign proteins expressed on the surface of cells to be targeted and destroyed. Activity of this defensive negative-feedback system may outpace the offensive function of the system, which may increase the probability that tumor cells evade detection and generate a cancerous growth.

Knowing how, and in what capacity, the various cell types are involved in the offensive attack against foreign cells and/or the defensive measures to limit immune system function may provide further insight into the development of cancerous tumors and/or autoimmune diseases. Thus, there exists a need for a diagnostic test to assess both the offensive and defensive immune function in an individual. There also exists a need to rapidly screen drugs for efficacy as immuno-modulating compounds and to indentify therapies for an individual based on that individuals' immune function.

### SUMMARY

Immunity plays a crucial role for the maintenance of good health and in defending against various diseases, ranging from a common cold to life-threatening illnesses. Although the last several decades have seen great advances in immunology, much of the understanding of the of how the immune system functions was derived from the in vitro experiments or experiments in animal models. Many of the therapies that are administered to patients are simple extrapolations from these experiments and not always effective across a wider variety of patients. Demand is growing for methods of characterizing each patient's immunity or immunological health by clinically applicable techniques.

In several embodiments, there is provided a method for determining whether a subject's immune function is directed toward offensive or defensive immune function, the method comprising, obtaining a first and a second sample containing leukocytes from a subject, exposing the first sample to an immune stimulating agent in a solvent, wherein the stimulating agent stimulates both offensive and defensive immune function, exposing the second sample to the solvent, quantifying the amount of one or more offensive immune function-related mRNAs in the first and second samples after exposing to the stimulating agent or solvent, thereby quantifying induction of offensive immune function as a ratio between the amount of the offensive immune function-related mRNAs quantified in the first and second samples, quantifying the amount of one or more defensive immune function-related mRNAs in the first and second samples after exposing to the stimulating agent or solvent, thereby quantifying induction of defensive immune function as a ratio between the amount of the defensive immune function-related mRNAs quantified in the first and second samples, wherein significantly more induction of offensive or defensive immune function-indicates that the subject's immune function is directed toward offensive or defensive immune function, respectively.

In some embodiments, the samples containing leukocytes are whole blood samples, which, in some embodiments, are optionally heparinized. In some embodiments, the stimulation need not occur immediately after obtaining the samples. Rather, the whole blood may be stored for up to about 24 hours prior to stimulation. In one embodiment, the stored sample is stored at room temperature. In another embodiment, the stored sample is stored in a refrigerated environment (e.g., less than room temperature, for example about 4 degrees Celsius)

In several embodiments, various stimulating agents are used, including, but not limited to one or more of recombinant interleukin-2, phytohemagglutinin, anti-T-cell receptor antibodies, heat aggregated IgG, lipopolysaccharide, and zymosan. In some embodiments, the exposure of the sample to the stimulating agent is less than 24 hours. In some embodiments, the exposure is for between 2 and 6 hours. In one embodiment, the exposure is for about 4 hours.

Functional categories may be used to categorize markers to be studied. In some embodiments, the one or more offensive immune function-related mRNAs are categorized as having immune recruiter function, immune killer function, or immune helper function. In some embodiments, the one or more offensive immune function-related mRNAs have immune recruiter function, and are selected from the group consisting of CCL2, CCL4, CCL8, CCL20, CXCL3, CXCL10, and Interleukin 8. In some embodiments, the one or more offensive immune function-related mRNAs have immune killer function, and are selected from the group consisting of granzyme B, perforin, TNFSF1, TNFSF2, TNFSF5, TNFSF6, TNFSF14, and TNFSF15. In some embodiments, the one or more offensive immune function-related mRNAs have immune helper function, and are selected from the group consisting of interleukin 2, interleukin 4, interferon gamma, and interleukin 17A.

The defensive immune function-related mRNAs are associated with suppression of offensive immune function. In some embodiments, the one or more defensive immune function-related mRNAs are selected from the group consisting of interleukin 10, transforming growth factor-beta, FoxP3, CD25, arginase, CTLA-4, and PD-1.

The methods described herein are optionally used when a subject has a cancer and the subject's immune function is determined as directed toward offensive or defensive immune function so as to identify a potentially efficacious an anti-cancer therapy. In such a use, a determination of the subject's immune function as directed toward offensive immune function indicates the likely efficacy of a cancer immunotherapy regimen. In some embodiments, a determination of the subject's immune function as directed toward defensive immune function indicates the likely efficacy of an anticancer regimen that does not involve an immune-based mechanism of action.

In several embodiments, the methods disclosed herein are used for predicting the efficacy of an anti-cancer therapeutic regimen based on the immune function of a subject, wherein a determination that the subject's immune function is directed toward offensive immune function indicates the likely efficacy of a anti-cancer immunotherapy regimen, and wherein a determination that the subject's immune function is directed toward defensive immune function indicates the likely efficacy of an anticancer regimen that does not involve an immune-based mechanism of action.

In several embodiments, there is provided a method for determining whether a subject's immune function is directed toward offensive or defensive immune function, the method comprising obtaining first and second samples containing leukocytes from the subject, exposing the first sample to an agent in a solvent, wherein the agent stimulates both offensive and defensive immune function, exposing the second sample to the solvent, incubating the exposed first and second samples, quantifying the amount of one or more defensive immune function-related mRNAs in each of the first and second samples after exposing to the agent or solvent, thereby determining an amount of induction of the defensive-immune function related mRNAs as a ratio between the amount quantified in the first and second samples, quantifying the amount of one or more offensive immune function-related mRNAs in each of the first and second samples after exposing to the agent or solvent, thereby determining an amount of induction of the offensive-immune function related mRNAs as a ratio between the amount quantified in the first and second samples, calculating a ratio between induction of the offensive immune function related mRNAs and the defensive immune function related mRNAs, comparing the calculated ratio with a control ratio derived from a group of control subjects, wherein a significant increase in the calculated ratio over the control ratio indicates the subject's immune function is directed toward offensive immune function and a significant decrease indicates the subject's immune function is directed toward defensive immune function.

In some embodiments, the offensive immune function related mRNAs comprise mRNAs encoding a marker of cytotoxic function. In some embodiments, the defensive immune function related mRNAs comprise mRNAs encoding a marker of myeloid derived suppressor cells, such as for example, arginase or FoxP3.

In several embodiments there is provided a method for identifying a drug for administration with a stimulator of both offensive and defensive immune function, wherein the administered drug inhibits one of the offensive or defensive immune functions, the method comprising quantifying the in vitro induction of expression of one or more offensive immune function associated markers in whole blood by measuring the expression of the offensive markers in the presence and absence of the drug (which can be in the presence or absence of the stimulator), quantifying the in vitro induction of expression of one or more defensive immune function associated markers in whole blood by measuring the expression of the defensive markers in the presence and absence of the drug (which can be in the presence or absence of the stimulator), and determining a difference between the induction of offensive immune function associated markers and the induction of defensive immune function associated markers in the presence and absence of the drug, wherein the drug is identified as for administration with the stimulator based on its net effect on the offensive and defensive markers.

In some embodiments, the in vitro induction comprises contacting the whole blood with the stimulator for a period of time sufficient to induce one or more of the offensive and the defensive immune function associated markers. In some embodiments, the offensive immune function associated markers comprise one or more of CD16, granzyme B, TNF-alpha, interferon gamma, and members of the tumor necrosis factor superfamily and the defensive immune function associated markers comprise one or more of CD25, FoxP3, CTLA4, GARP, IL 17, and arginase. In one embodiment, the stimulator of offensive and defensive immune function is interleukin 2. In one embodiment, the drug that wither offensive or defensive immune function induces expression of one or more offensive immune function associated markers to a greater degree that the drug induces the expression of one or more defensive immune function associated markers (e.g., it preferentially induces offensive function). The increase is optionally determined relative to a control sample that is stimulated with a solvent, which is the solvent used to deliver the drug. The administration of the drug is optionally prior to or concurrent with administration of the stimulating agent.

In several embodiments there is provided a method of characterizing both offensive and defensive immune function comprising obtaining at least two aliquots of heparinized whole blood, exposing the first aliquot to an agent that stimulates both offensive and defensive immune function, exposing the second aliquot to the solvent of the agent, incubating these aliquots for less than 24 hours, quantifying the amount of one or more mRNAs encoding TNFSF, granzyme B, perforin, CD16, INFgamma, or other genes representing the cytotoxic functions of leukocytes quantifying the amount of one or more mRNAs encoding FoxP3, CD25, or other genes representing the marker of regulatory T cells, calculating the ratio between expression of the markers of cytotoxic functions and the markers of regulatory T cells in both the first and second aliquots, and comparing these ratios with that derived from a group of control subjects.

The method optionally further comprises obtaining two additional aliquots of heparinized whole blood, exposing the first additional aliquot to an agent that stimulates both offensive and defensive immune function, exposing the second additional aliquot to the solvent of the agent, incubating these aliquots for less than 24 hours, quantifying the amount of one or more mRNAs encoding arginase or other genes representing the marker of myeloid derived suppressor cells, calculating the ratio between expression of the markers of cytotoxic functions and the markers of myeloid derived suppressor cells in both the first and second additional aliquots, and comparing these ratios with that derived from a group of control subjects.

### BRIEF DESCRIPTION OF THE FIGURES

FIGS. 1A-1D depicts the change over time in mRNA levels encoding various immune markers following IL-2 stimulation.

FIG. 2 depicts the dose response induction of immune markers to IL-2. A “*” indicates statistical significance (p<0.05) compared with solvent control.

FIGS. 3A-3J depicts the data used for drug screening.

FIGS. 4A-4J depict data related to induction of Offensive or Defensive immune markers used in drug screening.

FIGS. 5A-5D depict a rapid high throughput protocol.

### DETAILED DESCRIPTION

In several embodiments described herein, methods are provided for the ex vivo characterization of the offensive and defensive immune response of a host. As used herein, the term “offensive” shall refer to the overall immune response and cellular components associated therewith mounted against an infection, a foreign pathogen, a tumor or the like. In some instances the term “killer” is used interchangeably with “offensive.” As used herein, the term “defensive” shall refer to the overall immune response and cellular components associated therewith that serve to limit the activity of the activated offensive immune system. In some instances the term “suppressor” is used interchangeably with “defensive.”

In several embodiments, the methods involve collection of peripheral whole blood from a host and the use of a stimulating agent or agents to induce one or more of a panel of markers associated with the offensive or defensive immune response. In some embodiments, isolated leukocytes may optionally be used. In several embodiments, measurement of the mRNA encoding one or more of each of the offensive or defensive markers is used to characterize the overall immune response of a host. In still other embodiments, the characterization of the expression of such offensive or defensive markers is used to screen drugs for potential efficacy as either an immunosuppressant drug or an anti-cancer drug (e.g., immuno-modulating drugs).

The immune system comprises a variety of cell types having a variety of functions, which work together in concert to mount an attack on foreign bodies, thereby protecting the host from infection, tumorogenesis, etc. The main categories of function include, but are not limited to, recruitment function, killer function, suppressor (of killer) function, and helper function, as well as a variety of auxiliary functions, e.g., antigen presentation, regulation of angiogenesis, pain modulation etc.

Recruitment function is essential for the proper function of the immune system. In the event of an infection, tumor formation, etc., immune cells must be mobilized from various parts of the body, including the whole blood, bone marrow, and lymphatic system, among others, in order to properly recognize and defend the host from an immune challenge. In some embodiments, chemokines function to recruit other immune cells to the local area of inflammation or tumor formation. In essence, having an host of cells that can kill or disable unwanted foreign cells is useless if those cells are not properly instructed on where to go to function. Recruiter function is provided, in some embodiments, by chemokines or other chemotactic molecules. In some embodiments, chemokines of a particular motif function to recruit other immune molecules. For example, in several embodiments, CCL molecules, such as CCL-2, CCL-4, CCL-8, or CCL-20 are involved in recruiting other immune cells. In other embodiments, CXCL molecules, such as CXCL-3 or CXCL-10 are involved. In some embodiments, other chemokine effectors, whether C-C or C-X-C motif or another variety, are involved.

Once the recruiter cells have brought other types of immune cells to the proper location, the other types of cells can perform their designated function, which in some embodiments, is to kill the target cell(s). In some embodiments, the killing function is realized by induction of apoptosis in the target cell. For example, when the target is a tumor, one or more cells having killer function (e.g., expressing certain molecules involved in apoptosis) are recruited to the target site. In some embodiments, such killer cells express one or more of molecules such as Granzyme B, perforin, TNFSF1 (lymphotoxin), TNFSF2 (TNF-alpha), TNFSF 5 (CD40 ligand), TNFSF6 (Fas ligand), TNFSF14 (LIGHT), TNFSF 15 (TL1A), and/or CD16. As such, the recruitment of these cells to the target site initiates a cascade that results in the destruction of the target cells, and thus realizes the goal of the offensive immune system, e.g., destruction and/or removal of a foreign body or cell.

As discussed herein, the defensive immune system comprises a series of signals and molecules that function to limit the activity of the offensive immune system (e.g., prevent overactive offensive function, which could lead to autoimmune disorders). Cells having defensive function can be recognized by markers including, but not limited to, IL10, TGF-beta, (forkhead box p3) FoxP3, CD25, arginase, CTLA-4, and /or PD-1. Such cells are an important balance on activity of the offensive immune system and are important to ensure proper overall immune function.

Additional cells types are involved in the functioning of both the offensive and defensive immune system. Helper T-cells (Th cells) are a sub-group of lymphocytes function to establish and maximize the capabilities of the immune system. Unlike the cells described above, Th cells lack cytotoxic or phagocytic activity. Th cells are, however, involved in activating and directing other immune cells such as the cytotoxic T cells (e.g., the killer cells described above). Th cells are divided into two main subcategories (Th1 or Th2) depending on, among other factors, what cell type they primarily activate, what cytokines they produce, and what type of immune stimulation is promoted. For example, Th1 cells primarily partner with macrophages, while Th2 cells primarily partner with B-cells. Th1 cells produce interferon-gamma, TNF-beta, and IL-2, while Th2 cells product IL1, IL5, IL6, IL10 and IL13. Markers of the subsets of Th cells are known and can be used to identify the induction of certain Th cell subtypes in response to stimulation. For example, the induction of IL2 or IFNG represent responses to stimulation by Th1 cells, while induction of IL4 or IL10 represent responses to stimulation by Th2 cells. Other subtypes, such as Th17 are represented by other markers, such as IL17 (see e.g., Tables 5 and 6).

Finally, a variety of other functions are useful to study when characterizing immune status or function of a subject. For example, antigen presentation (measured by GMCSF), proliferation of B-cells (measured by IGH2), angiogenesis, which is often occurs in tumor formation due to increased blood flow demands (measured by VEGF), and pain (measured by POMC). These general categories described above can be used to categorize interpret data generated by stimulation of whole blood in order to characterize a subject's immune response, which is described in more detail below.

Building on the general functional categories described above, offensive immune function, such as the function of NK cells and cytotoxic T cells is important for destruction of cancerous cells and combating infections and/or inflammation. Due to their ability to potentially kill both unwanted target cells as well as normal endogenous cells, NK cells possess two types of surface receptors, activating receptors and inhibitory receptors. Together, these receptors serve to balance the activity of, and therefore regulate, the cytotoxic activity of NK cells. Activating signals are required for activation of NK cells, and may involve cytokines (such as interferons), activation of FcR receptors to target cells against which humoral immune responses have been mounted, and/or foreign ligand binding to various activating NK cell surface receptors. Targeted cells are then destroyed by the apoptotic mechanism described above.

Similarly, cytotoxic T cells also require activation, thought to be through a two signal process resulting in the presentation of a foreign (e.g., non-self) antigen to the cytotoxic T cells. Once activated, cytotoxic T cells undergo clonal expansion, largely in response to interleukin-2 (IL-2), a growth and differentiation factor for T cells. Cytotoxic T cells function somewhat similarly to NK cells in the induction of pore formation and apoptosis in target cells.

In addition to the immune attack on foreign cells, defensive immune function develops, which is believed to be moderated by T-reg and MDSCs, and inhibits offensive immune function. Developing in the thymus, many T-reg express the forkhead family transcription factor FoxP3 (forkhead box p3). FoxP3 expression appears to be required for T-reg development and population expansion and may also be a controlling factor in a genetic program defining the T-reg fate. In many disease states, particularly cancers, alterations in T-reg numbers, particularly those T-reg expressing Foxp3, are found. For example, patients with tumors have a local relative excess of Foxp3 positive T cells which inhibits the body's ability to suppress the formation of cancerous cells.

MDSCs also are effectors of the defensive immune response. While MDSCs do not appear to destroy offensive T cells, they do alter how cytotoxic T cells behave. MDSCs secrete arginase (ARG), a protease that breaks down the amino acid arginine. Lymphocytes, including cytotoxic T cells and NK cells are indirectly dependent on arginine for activation. Thus, the secretion of ARG by MDSCs limits the activation of NK cells and cytotoxic T cells, thereby promoting the defensive immune response.

However, as a result of this self-limiting regulation by T-reg and MDSCs, defensive immune function has the potential to become the dominant scheme in a local tissue environment. As a result, the stimulation of offensive immune function (as developed by cancer vaccine treatment or adaptive immunotherapy) may fail to function sufficiently to completely eradicate tumor cells. As a result, a tumor cell may escape the immune system and metastasize into a tumor.

Thus, the characterization of offensive and defensive immune function in an individual may be critically important, as domination by the offensive system may promote auto-immunity and domination by the defensive system may be pro-cancerous. Furthermore, the expression profiles of offensive and defensive-associated immune markers may be useful to screen drugs for their efficacy as anti-cancer medications or immunosuppressant agents.

For example, the expression profiles of various markers can be used to identify therapies of a particular variety that may be most efficacious for a particular subject. The stimulation of whole blood by a variety of markers enables a determination of what cell types are responsive to a particular type of stimulus. As discussed herein, stimulants include, but are not limited to, IL2 (a general cellular immune modulator), PHA (a general T-cell modulator), anti-T-cell receptor antibodies (specific stimulator of T-cells), HAG (stimulant for leukocytes having the FcR receptor), and LPS or zymosan (activators of the Toll-like receptor, associated with general bacterial immune responses). When used to stimulate whole blood and measure the induction of expression of various markers from the general functional categories described herein (e.g., recruiter or killer), the pattern of induction can be used to identify potential therapies that are particularly effective for a given subject. As a non-limiting example, if a sample of blood from a patient having cancer is stimulated and one or more markers of offensive (e.g., killer) function are increased, this initially suggests that an immunotherapy based cancer treatment may be effective for this subject. The strength of the initial suggestion may be increased based on the expression levels of markers from other categories. For example, if the induction of one or more offensive markers is associated with a stable (e.g., little or no change) in suppressor function, this further suggests that an immunotherapy based cancer treatment would likely be effective, given the increase in offensive function without a coordinate increase in defensive function that would limit the efficacy of the offensive arm of the immune system. In other words, the increased gap in function, as represented by expression levels, between the offensive and defensive immune systems indicates that a therapy exploiting the increased offensive activity would be effective. On the other hand, for example, if stimulation of a patient's whole blood yields an increase in offensive markers as well as in defensive markers (e.g., no net change in function between the systems despite the increase), these results would suggest a non-immune-based therapy may be more efficacious for this subject. This is because the lack of a net change in function between the offensive and defensive systems suggests that both systems are upregulated in function, and that the offensive system is not likely to be sufficiently dominant to render an immune-based therapy particularly effective. In such a context, therapies such as radiation, surgery, or chemotherapy may be more effective. In several embodiments, additional information from expression levels of markers in other functional categories may support or refute other data related to the potential efficacy of a particular type of therapy. For example, stimulation of the whole blood of a cancer patient that results in increased recruiter marker expression in conjunction with increased offensive marker expression and little or no change in defensive marker expression further supports the potential efficacy of an anti-cancer therapy that is immune-based. This is because the increased recruiter activity is likely to further enhance the increased offensive function by enabling the cells of the offensive immune system to be recruited, for example, more quickly, in greater numbers, and/or over a longer period of time. In some embodiments, expression changes in functional categories are evaluated and used to determine a potentially optimal therapy, while in other embodiments, individual markers from within a category are used to determine a potentially optimal therapy.

Numerous methods for assessing the expression of markers of interest are available. For example, flow cytometric analysis may allow the identification of NK cells, cytotoxic T cells, T-reg, and MDSCs by staining appropriate marker proteins. However, such an assay system is not capable of analyzing each cell's function. Therefore, several embodiments involve the ex vivo induction and measurement of offensive and defensive-associated immune response mRNAs as a diagnostic test to characterize a patient's overall immune response.

Moreover, many prior experiments directed to determining expression of immune system activity and/or markers of activity have been done in isolated leukocyte preparations. Such isolated populations are often preferred because the variety of lymphocytes in whole blood may preclude detection of induction of a specific mRNA in a small subset of lymphocytes. Moreover, with numerous complex biochemical interactions between the multiple types of lymphocytes, there is the possibility that use of a whole blood preparation inhibits or modifies the induction and measurement reactions. Furthermore, stimulatory agents, such as IL-2 and zymosan, which are used in several embodiments, may interact with plasma proteins or plasma factors, and thereby exhibit decreased or reduced induction activity. However, when used as presented in several embodiments as described herein, whole blood unexpectedly produces reproducible, accurate, and physiologically relevant results that allow the characterization of both offensive and defensive immune markers and screening of drugs of immune-modulating efficacy.

In several embodiments, whole blood is collected from mammals, preferably humans. In several preferred embodiments, the collected whole blood is heparinized upon collection. In several embodiments, the collected whole blood is stored at 4° C. until the stimulation protocol (described below). While preferred embodiments employ whole blood, in other embodiments, blood cells separated from plasma may also be used, as well as isolated leukocyte preparations.

In several preferred embodiments of the method, the blood is aliquoted into small volumes (approximately 40-100 microliters (μL)), each of which is treated (i.e., induced or stimulated) with either a stimulating agent carried in a solvent or a control agent. In some embodiments, the control agent induces little or no response in the blood samples. In certain embodiments, the control agent is the same solvent used to carry the stimulating agent. In certain embodiments, the control agent is phosphate-buffered saline (PBS), while in other embodiments, the control agent is dimethyl sulfoxide (DMSO). In several embodiments, recombinant IL-2 (rIL-2) is used as a stimulating agent for both offensive and defensive immune markers. IL-2 is often used clinically as an agent to augment offensive immune response. However, this clinical effort also fails on some occasions, as IL-2 can also simultaneously upregulate the defensive immune system. Thus, in order to develop a more complete analysis of both major portions of the immune system, IL-2 is a preferred stimulatory agent in certain embodiments. In other embodiments, zymosan, a ligand of the toll-like receptor type 2 (TLR-2), is used as a stimulating agent for both offensive and defensive markers. In some embodiments, other known immune-stimulating agents are used. In yet other embodiments agents known to stimulate particular offensive and/or defensive immune markers are used.

In several embodiments, the stimulating agents induce the expression of one or more offensive or defensive immune markers, as measured by the amount of mRNA encoding said markers. Offensive markers include, but are not limited to, CD16 (surface marker of NK cells); granzyme B (inducer of rapid apoptosis); perforin (cytolytic protein that functions to lyse cells); TNFSF1 (lymphotoxin, functions to enhance phagocytic cell binding to a target cell); TNFSF2 (TNF-alpha; inducer of slow apoptosis); TNFSF5 (CD40 ligand, operates to activate antigen presenting cells and macrophages); TNFSF6 (Fas ligand, inducer of apoptosis); TNFSF14 (LIGHT; induces T-cell proliferation and apoptosis of tumor cells); TNFSF15 (inducer of apoptosis). Defensive immune markers include, but are not limited to IL10 (down-regulator of Th1 cytokines); TGF-beta (blocks lymphocyte activation); CD25 (surface marker of T-reg); FoxP3 (T-reg marker); CTLA4 (Cytotoxic T-lymphocyte antigen); GARP (glycoprotein A repetitions predominant); IL17 (putative negative regulator of T cell activation); ARG (arginase, marker of MDSC); and PD-1 (programmed death 1, negative regulator of T-cell responses).

In several embodiments of the method, induction of offensive or defensive immune markers is accomplished by mixing a small aliquot of a blood sample with either a control agent in triplicate, or one of the stimulating agents in triplicate. The mixture is then incubated at 37° C., for a period of time sufficient for induction of the offensive or defensive immune markers to occur. In some embodiments, the incubation time is approximately 4 hours. In certain embodiments, the incubation time may be greater than 4 hours. In certain embodiments, the incubation time is approximately 24 hours. In certain other embodiments, the incubation time may be less than 4 hours. After the appropriate incubation period, all the blood samples are stored at −80° C. until further analysis.

In several embodiments, a small volume of the previously stimulated blood from each sample is processed to allow determination of the levels of mRNA encoding one or more offensive or defensive immune markers in the blood. In some embodiments, the levels of mRNA encoding one or more offensive or defensive immune markers will change significantly in response to the stimulating agent. To determine these mRNA levels, the erythrocytes and blood components other than leukocytes are removed from the blood sample. In preferred embodiments, the leukocytes are isolated using a device for isolating and amplifying mRNA. Embodiments of this device are described in more detail in U.S. patent application Ser. Nos.: 10/796,298, 11/525,515, 11/376,018, 11/803,593, 11/803,594, and 11/803,663, each of which is incorporated in its entirety by reference herein.

In brief, certain embodiments of the device comprise a multi-well plate that contains a plurality of sample-delivery wells, a leukocyte-capturing filter underneath the wells, and an mRNA capture zone underneath the filter which contains immobilized oligo(dT). In certain embodiments, the device also contains a vacuum box adapted to receive the filter plate to create a seal between the plate and the box, such that when vacuum pressure is applied, the blood is drawn from the sample-delivery wells across the leukocyte-capturing filter, thereby capturing the leukocytes and allowing non-leukocyte blood components to be removed by washing the filters. In other embodiments, other means of drawing the blood samples through out of the sample wells and through the across the leukocyte-capturing filter, such as centrifugation or positive pressure, are used. In preferred embodiments of the device, leukocytes are captured on a plurality of filter membranes that are layered together. In several embodiments, the captured leukocytes are then lysed with a lysis buffer, thereby releasing mRNA from the captured leukocytes. The mRNA is then hybridized to the oligo(dT)-immobilized in the mRNA capture zone. Further detail regarding the composition of lysis buffers that may be used in several embodiments can be found in U.S. patent application Ser. No. 11/376,018, which is incorporated in its entirety by reference herein. In several embodiments, cDNA is synthesized from oligo(dT)-immobilized mRNA. In preferred embodiments, the cDNA is then amplified using real time PCR with primers specifically designed for amplification of infection-associated markers. Primers that are used in such embodiments are shown in Table 1. Further details about the PCR reactions used in some embodiments are also found in U.S. patent application Ser. No. 11/376,018.

TABLE 1
Primer Sequences for RT-PCR Amplification

| | | | SEQ ID | | SEQ ID |
|---|---|---|---|---|---|
| Target | Class | FWD Sequence (5′-3′) | NO. | REV Sequence (3′-5′) | NO. |
| ACTB | Control | CCTGGCACCCAGCACAAT | 1 | GCCGATCCACACGGAGTACT | 2 |
| B2M | Control | TGACTTTGTCACAGCCCAAG ATA | 3 | AATGCGGCATCTTCAAACCT | 4 |
| CCL2 | Recruiter | CCATTGTGGCCAAGGAGATC | 5 | TGTCCAGGTGGTCCATGGA | 6 |
| CCL4 | Recruiter | GGTATTCCAAACCAAAAGAAGCA | 7 | GTTCAGTTCCAGGTCATACACGTACT | 8 |
| CCL8 | Recruiter | AGAGCTACACAAGAATCACCAACATC | 9 | AGACCTCCTTGCCCCGTTT | 10 |
| CCL20 | Recruiter | GATACACAGACCGTATTCTTCATCCTAA | 11 | TGAAAGATGATAGCATTGATGTCACA | 12 |
| CXCL3 | Recruiter | GGAATTCACCTCAAGAACATCCA | 13 | GTGGCTATGACTTCGGTTTGG | 14 |
| CXCL10 | Recruiter | TCCACGTGTTGAGATCATTGC | 15 | TCTTGATGGCCTTCGATTCTG | 16 |
| IL8 | Recruiter | TGCTAAAGAACTTAGATGTCAGTGCAT | 17 | TGGTCCACTCTCAATCACTCTCA | 18 |
| Granzyme B | Offensive | GCGGTGGCTTCCTGATACAA | 19 | CCAAGGTGACATTTATGGAGCTT | 20 |
| PRF1 | Offensive | TCCTTGGCACCTGTGATCAG | 21 | CCATGATTCAGGTTGCATCTCA | 22 |
| TNFSF1 | Offensive | CAGCTATCCACCCACACAGATG | 23 | CGAAGGCTCCAAAGAAGACAGT | 24 |
| TNFSF2 | Offensive | CGAAGGCTCCAAAGAAGACAGT | 25 | CAGGGCAATGATCCCAAAGT | 26 |
| TNFSF5 | Offensive | CCACAGTTCCGCCAAACCT | 27 | CACCTGGTTGCAATTCAAATACTC | 28 |
| TNFSF6 | Offensive | TGGCAGCATCTTCACTTCTAAATG | 29 | GAAATGAGTCCCCAAAACATCTCT | 30 |
| TNFSF14 | Offensive | CGTCCGTGTGCTGGATGA | 31 | CATGAAAGCCCCGAAGTAAGAC | 32 |
| TNFSF15 | Offensive | TGCGAAGTAGGTAGCAACTGGTT | 33 | CCATTAGCTTGTCCCCTTCTTG | 34 |
| CD16 | Offensive | GTTTGGCAGTGTCAACCATC TC | 35 | AAAAGGAGTACCATCACCAAGCA | 36 |
| IL10 | Defensive | GCCATGAGTGAGTTTGACAT CTTC | 37 | GATTTTGGAGACCTCTAATTTATGTCCTA | 38 |
| TGFB1 | Defensive | CTGCTGAGGCTCAAGTTAAAAGTG | 39 | TGAGGTATCGCCAGGAATTGT | 40 |
| FOXP3 | Defensive | CACCTACGCCACGCTCATC | 41 | AAGGCAAACATGCGTGTGAA | 42 |
| CD25 | Defensive | CAGAAGTCATGAAGCCCAAGTG | 43 | GGCAAGCACAACGGATGTCT | 44 |
| CTLA4 | Defensive | CATGCCTCCTCTTCTTCCTTGA | 45 | GGAGGGTGCCACCATGACTA | 46 |
| PD-1 | Defensive | CTCAGCCGTGCCTGTGTTC | 47 | GGAAAGACAATGGTGGCATACTC | 48 |
| ARG | Defensive | AGACACCAGAAGAAGTAACTCGAACA | 49 | TCCCGAGCAAGTCCGAAAC | 50 |
| IL2 | Helper | GAACTAAAGGGATCTGAAACAACATTC | 51 | TGTTGAGATGATGCTTTGACAAAA | 52 |
| IL4 | Helper | CACAGGCACAAGCAGCTGAT | 53 | CCTTCACAGGACAGGAATTCAAG | 54 |
| INF-γ | Helper | GGAGACCATCAAGGAAGACATGA | 55 | GCTTTGCGTTGGACATTCAA | 56 |
| IL17 | Helper | GAAATCCAGGATGCCCAAATT | 57 | CGGTTATGGATGTTCAGGTTGA | 58 |
| GMCSF | Antigen | GGCCCCTTGACCATGATG | 59 | TCTGGGTTGCACAGGAAGTTT | 60 |
| | Presentation | | | | |
| IGH@ | Helper | CAGCCGGAGAACAACTACAAGAC | 61 | GCTGCCACCTGCTCTTGTC | 62 |
| VEGF | Angiogenesis | CGCAGCTACTGCCATCCAAT | 63 | TGGCTTGAAGATGTACTCGATCTC | 64 |
| POMC | Pain | ACGAGGGCCCCTACAGGAT | 65 | TGATGATGGCGTTTTTGAACA | 66 |
| GARP | Offensive | GACCTGATCTGCCGCTTCA | 67 | CCAGCGTGGTGAGGAGGAT | 68 |
| CD11a | Recruiter | GGAGATCCTCGTCCAAGTGATC | 69 | GAGGCGTTGCTGCCATAGAG | 70 |
| CD122 | Offensive | CATATTTACAACAGAGTACCAGGTAGCA | 71 | TTACCAAGAAATTCTTGTTCTTTTGG | 72 |

After the completion of PCR reaction, the mRNA (as represented by the amount of PCR-amplified cDNA detected) for one or more offensive or defensive immune markers is quantified. In certain embodiments, quantification is calculated by comparing the amount of mRNA encoding an offensive or defensive immune marker to a reference value. In other embodiments, the reference value is expression level of a gene that is not induced by the stimulating agent, e.g., a house-keeping gene. In certain such embodiments, beta-actin is used as the reference value. Numerous other house-keeping genes that are well known in the art may also be used as a reference value. In other embodiments, a house keeping gene is used as a correction factor, such that the ultimate comparison is the induced expression level of an offensive or defensive immune marker as compared to the same marker from a non-induced (control) sample. In still other embodiments, the reference value is zero, such that the quantification of the offensive or defensive immune markers is represented by an absolute number. In several embodiments a ratio comparing the expression of one or more offensive immune markers to one or more defensive immune markers is made.

In several other embodiments, offensive or defensive immune marker expression is measured in the presence of a drug (either a putative anti-cancer or immunosuppressant drug) both in the presence and in the absence of a stimulating agent. In such embodiments, the expression profiles may be used to predict the efficacy of a drug compound as an effective anti-cancer drug or as an effective immunosuppressant drug. In some embodiments, a drug compound will induce the expression of an offensive immune marker but not a defensive marker, which would promote the offensive immune system overall, thus making that drug compound a putative anti-cancer therapeutic. Likewise, in other embodiments, a drug may inhibit one or more defensive markers, which would promote the offensive immune system overall, thus making that drug compound a putative anti-cancer therapeutic. In some such embodiments, a drug that blocks a defensive immune marker, and hence reduces the defensive immune component could be co-administered with a therapeutic agent known to stimulate the offensive immune system (such as IL-2), thereby providing an enhance offensive immune response and increased likelihood of tumor cell elimination.

In contrast, in some embodiments, a drug compound may induce the expression of a defensive immune marker but not an offensive marker, which would promote the defensive immune system overall, thus making that drug compound a putative immunosuppressant. Likewise, in other embodiments, a drug may inhibit one or more offensive markers, which would promote the defensive immune system overall, thus making that drug compound a putative immunosuppressant.

In still other embodiments, a drug compound may not induce either offensive or defensive marker, or may induce both. In such embodiments, further dose-response study is performed to determine if a particular dose or exposure time categorizes a drug as a putative anti-cancer drug or putative immunosuppressant.

### EXAMPLES

Specific embodiments will be described with reference to the following examples which should be regarded in an illustrative rather than a restrictive sense.

### Example 1

### Characterization of Offensive and Defensive Immune Markers in Whole Blood Samples

Whole blood samples were collected from healthy human adults. Blood samples were heparinized when collected and placed into several individual tubes for an induction time course study. Eighteen equal volume aliquots from each tube were then stimulated with either phosphate buffered saline (PBS), rIL-2 at 100 ng/mL, or phytohemagglutinin (PHA). The aliquots were incubated at 37° C. for 0, 1, 2, 4, 8, or 20 hours. After incubation, samples were stored at −80° C. until analysis. Each sample was stimulated and analyzed in triplicate. mRNA encoding beta-actin, IL-2, IL-2 receptor type a (CD25) and IL-2 receptor type b (CD122) were measured according to the methods described in Mitsuhashi M, et al., Clin Chem 52:634-642 (2006), which is incorporated in its entirety by reference herein.

Briefly, 96-well filterplates were assembled with leukocyte reduction membranes (Leukosorb; Pall) and placed over oligo(dT)-immobilized collection plates. 150 μL of 5 mmol/L Tris (pH 7.4) was applied to wet the filter membranes. After centrifugation at 120 g for 1 min at 4° C. to remove the Tris solution from the membranes, 50 μL of the stimulated whole blood samples was applied to each well and immediately centrifuged at 120 g for 2 min at 4° C. The wells were then washed once with 300 μL of phosphate-buffered saline. After centrifugation at 2000 g for 5 min at 4° C. to remove the saline solution, 60 μL of stock lysis buffer [5 g/L N-lauroylsarcosine, 4× standard saline citrate, 10 mmol/L Tris-HCl (pH 7.4), 1 mmol/L EDTA, 1 mL/L IGEPAL CA-630 (substitute of NP-40), 1.79 mol/L guanidine thiocyanate (all from Sigma)], supplemented with 10 mL/L 2-mercaptoethanol (Bio-Rad), 0.5 g/L proteinase K (Pierce), 0.1 g/L salmon sperm DNA (5 Prime Eppendorf/Brinkman), 0.1 g/L Escherichia coli tRNA (Sigma), 5 nmol/L each of the specific reverse primers, and 10¹⁰ molecules/L of synthetic RNA34 (as external control), was added to each well of the filterplates. The plates were then incubated at 37° C. for 10 min, placed over oligo(dT)-immobilized collection microplates (GenePlate; RNAture), and centrifuged at 2000 g for 5 min at 4° C. After overnight storage at 4° C., the microplates were washed 3 times with 100 μL of plain lysis buffer and then 3 times with 150 μL of wash buffer [0.5 mol/L NaCl, 10 mmol/L Tris (pH 7.4) 1 mmol/L EDTA] at 4° C.

cDNA was synthesized directly in each well by addition of 30 μL of buffer containing 1× reverse transcription buffer [50 mM KCl, 10 mM Tris-HCl (pH 8.3), 5.5 mM MgCl₂, 1 nL/μL Tween 20], 1.25 mM each deoxynucleoside triphosphate, 4 units of rRNasin, and 80 U of MMLV reverse transcriptase (Promega; without primers) and incubation at 37° C. for 2 h. From each 30 μL reaction, 4 μL of cDNA was transferred directly to 384-well PCR plates, and 5 μL of TaqMan universal master mixture (Applied Biosystems) and 1 μL of 5 μM each of the forward and reverse primers for an infection-associated marker or beta-actin (see Table 1) were added. Primer sequences used are shown in Table 1 above. Primer sequences for ACTB (β-actin) were also published previously (Mitsuhashi M, et al. Pharm Res. 25:1116-1124, 2008), which is incorporated in its entirety by reference herein. PCR was carried out in a PRISM 7900HT (Applied Biosystems), with 1 cycle of 95° C. for 10 min followed by 45 cycles of 95° C. for 30 s, 55° C. for 30 s, and 60° C. for 1 min. Each gene was amplified in separate wells. The cycle threshold (Ct), i.e., the cycle at which certain amounts of PCR products (based on fluorescence) were generated, was determined with analytical software (SDS; Applied Biosystems). The Ct of each mRNA was subtracted with that of ACTB to calculate ΔCt, and % ACTB was calculated by 2^{-ΔCt}×100.

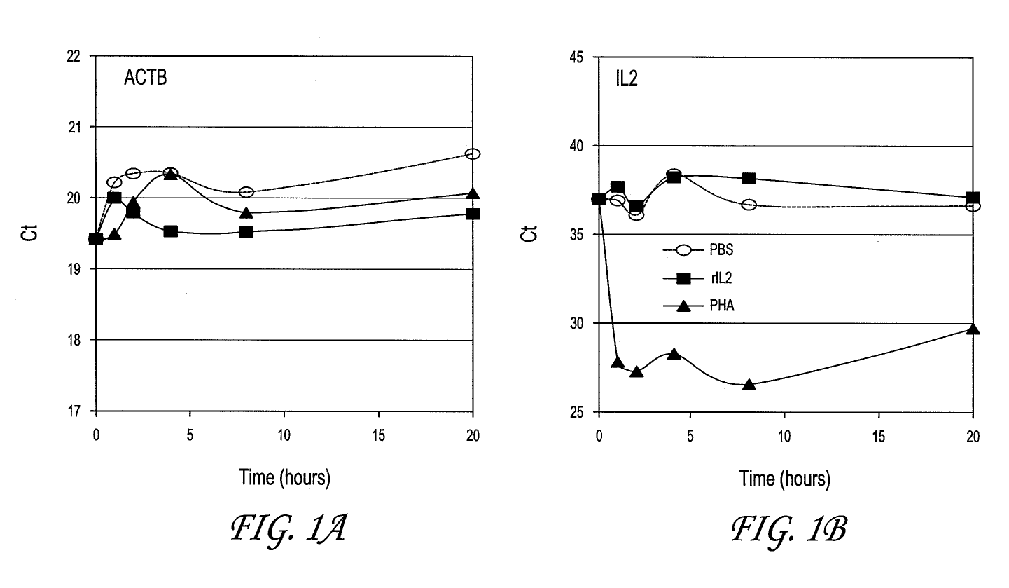

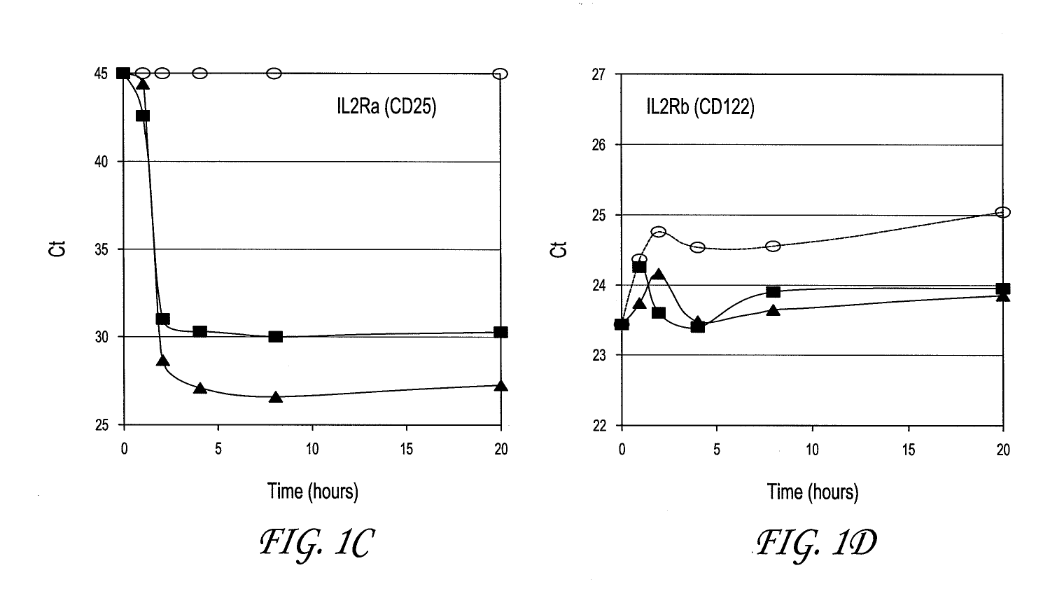

As shown in FIG. 1A, stimulation of whole blood does not induce beta actin expression, regardless of the stimulating agent used. FIG. 1B serves as a positive control for the stimulation assay, as IL-2 is not self-inducing, but PHA, a known stimulator of inflammatory and immune responses robustly increased the expression of IL-2. FIG. 1C demonstrates that the induction of a defensive immune marker CD25 occurs subsequent to stimulation by either rIL-2 or PHA (an inducer of IL-2). This also demonstrates the cascade effect that IL-2 stimulation has on both the offensive and defensive aspects of the immune system.

Additional aliquots of the whole blood samples were used for a dose-response study. Seven aliquots were stimulated for four hours with rIL-2 at 100 ng/mL. After incubation, samples were stored at −80° C. until analysis. Each sample was stimulated and analyzed in triplicate. mRNA encoding beta-actin, IL-2, IL-2 receptor type a (CD25), IL-2 receptor type b intronic sequence (CD122), IL-2 receptor type b exon sequence (CD122), granzyme B, TNF-alpha, and interferon-gamma were measured according to the methods described above.

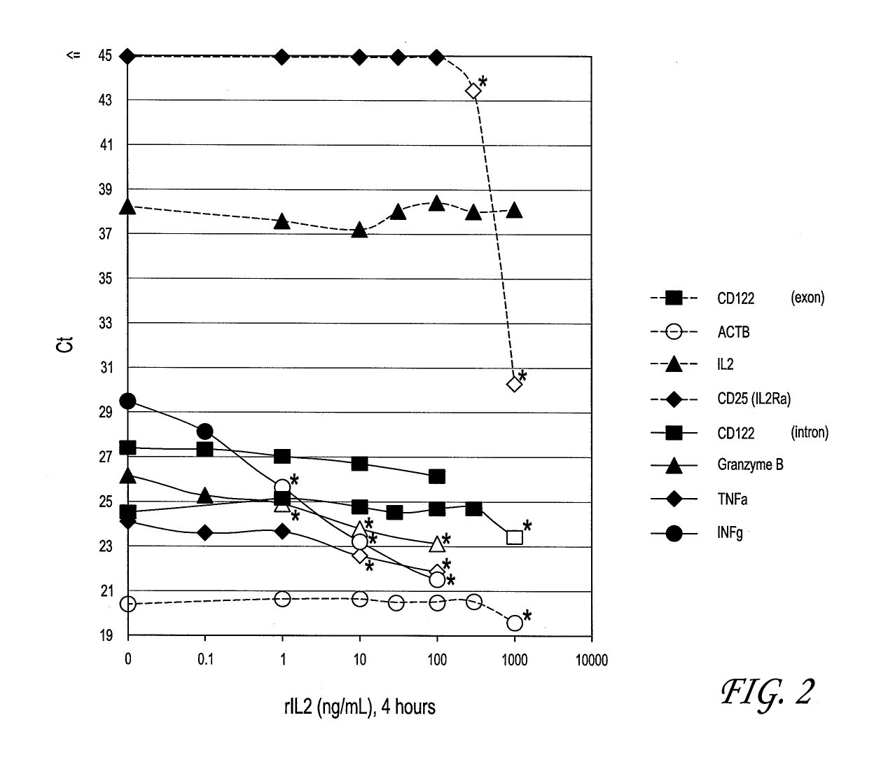

As shown in FIG. 2, offensive and defensive markers may respond differently to a given concentration of stimulatory agent. Based on the effect on induction of offensive markers and the time course depicted in FIG. 1, stimulation with 100 ng/mL rIL-2 for 4 hours were the conditions chosen for a panel of stimulation experiments. It shall be appreciated that greater or lesser concentrations of a stimulatory agent may be used in other embodiments. Likewise, greater or lesser times for stimulation may be used, for example 1-2 hrs, 2-4 hours, 4-6 hours, 6-8 hours, 8-10 hours, 10-12 hours, 12-18 hours, 18-24 hours, and overlapping ranges thereof.

Induction of expression of a panel of offensive or defensive markers was studied in response to stimulation of whole blood by rIL-2 or zymosan. Samples were generally prepared and stimulated as described above, with rIL-2 used at 100 ng/ml and zymosan used at 1.5 mg/mL and stimulation was for four (4) hours at 37° C.

As shown in Table 2, stimulation with rIL-2 induced statistically significant (P<0.05) increases in all offensive markers tested in the panel. While TNFα, CD16, and Granzyme B were induced approximately 4-8 fold over control samples, IFNγ was induced 260 fold over control samples. rIL-2 also induced statistically significant increases in several defensive markers, namely FoxP3, CD25, and IL 17. Zymosan induced a statistically significant increase in arginase. These results demonstrate that the methods of certain embodiments of the invention as disclosed herein unexpectedly allow the detection of increased expression of both offensive and defensive markers of immune function, in contrast to the suggestion and conventional wisdom in the related art that isolated leukocyte preparations are needed. These data indicate that stimulation conditions used in several embodiments induce the expression of various offensive and defensive markers well above the threshold for detection (background noise) of the assay and provide a rapid, simple, and reliable method of characterizing the offensive and defensive immune response of an individual.

TABLE 2
Leukocyte Function by Ex Vivo Stimulation

| | | rIL-2 (100 ng/mL) | | | Zymosan (TLR-2; 1.5 mg/mL) | | |
|---|---|---|---|---|---|---|---|
| | mRNA | Avg. | S.D. | p-value | Avg. | S.D. | p-value |
| Control | ACTB | 0.9088 | 0.0756 | 0.247 | 0.63 | 0.011 | 2 × 10⁻⁶* |
| Offensive | TNFα | 4.73 | 0.431 | 5 × 10⁻⁴* | 11 | 0.886 | 8 × 10⁻⁵* |
| | CD16 | 6.1303 | .03566 | 5 × 10⁻⁴* | | | |
| | Granzyme B | 8.2773 | 1.5272 | 2 × 10⁻⁴* | | | |
| | IFNγ | 260.48 | 78.401 | 2 × 10⁻⁵* | 5.948 | 6.978 | n.s. |
| Defensive | FoxP3 | 3.123 | 0.8372 | 0.029* | | | |
| | CD25 | 16.035 | 0.999 | 5 × 10⁻⁴* | | | |
| | CTLA4 | 1.0843 | 0.1799 | n.s. | | | |
| | (1^{st} Primer) | | | | | | |
| | GARP | 0.7404 | 0.14 | n.s. | | | |
| | IL17 | 18.315 | 7.2536 | 0.005* | | | |
| | ARG | | | | 3.618 | 0.846 | 0.005* |

*Fold increases >2 are statistically significant based on a p-value of <0.05

### Example 2

### Induction of Offensive and Defensive Immune Markers as Method of Drug Screening

Several embodiments of the present invention are used to screen candidate drug compounds based on the ability of the compounds to induce expression of offensive or defensive immune markers. In some embodiments, such a drug screening assay would increase the efficiency of identifying a compound that could be co-administered with IL-2 in an anti-cancer clinical setting, the compound ideally blocking increases in expression of defensive immune markers. Thus, IL-2 stimulation would induce offensive immune responses to putative cancer cells, and the candidate compound would block the IL-2 induced negative feedback defensive immune responses, thereby limiting the self-induced down regulation of the offensive response. In this manner, the offensive immune response is effectively potentiated by the lack of (or reduction of) defensive immune responses.

Similarly, several embodiments are used to screen for potential immunosuppressant compounds. Potential efficacious compounds are those that inhibit the induction of offensive immune markers and do not effect (or increase) defensive immune markers, thereby promoting the activity of the defensive immune system over those of the offensive system. Such a response would allow for suppression of the endogenous immune response and potentially provide benefit to transplant patients or patients suffering from autoimmune disorders.

While certain compounds may exhibit likely efficacy for suppressing (or stimulating) offensive or defensive immune function, it shall be appreciated that potential efficacy may not be shown at a single dose tested. Thus, in some embodiments, dose-response testing is also performed to determine whether a potential compound with limited efficacy at a first dose has enhanced efficacy (as offensive or defensive immune stimulator) at different doses, which may be higher or lower than the first concentration.

To screen potential compounds, heparinized whole blood was preincubated with various potential immune inhibitor compounds (all 10 mM in final concentration) or solvent control (DMSO) for 1 hour in a single tube for each treatment, then stimulated with PBS or rIL-2 (100 ng/ml in final concentration) in triplicate for additional 4 hours. mRNA was then quantified as described above. Potential immune inhibitor compounds tested are shown in Table 3.

TABLE 3
Identifier and Target of Inhibitors Tested

| Inhibitor Identifier | Target |
|---|---|
| SB239063 | MAP kinase |
| PD98059 | MAPKK |
| GSK-3b | GSK-3b |
| JNK | JNK |
| rapamycin | mTOR |
| everolimus | mTOR |
| Jak inhibitor 1 | Jak |
| CsA (cyclosporin A) | calcineurin |
| Tacrolimus | calcineurin |
| PD 153035 | EGFR |
| E804 | src, cdk/cyclin |
| Jak1 | Jak1 |
| Jak2 | Jak2 |
| Ly (=Ly294002) | PI3 kinase |
| PP2 | p56lck |
| SHP | SHP1/2 PTPase |
| STAT3p (STAT3 inhibitor peptide) | STAT3 |
| STAT3w (STAT3 inhibitor III, WP1066) | STAT3 |
| STAT3w (STAT3 inhibitor V) | STAT3 |
| STAT5 | STAT5 |
| AG490 | EGFR |
| U0126 | MEK1 |

Beta-actin expression served as a control for the expression assay. Offensive immune markers characterized included tumor necrosis factor superfamily (TNFSF) 1 (lymphotoxin), 2 (TNFα), 5 (CD40L), 6 (FasL), 8 (CD30L), 9 (CD137L), 14 (LIGHT), and 15 (TL1A). Chemokines characterized included CCL2, CCL3, CCL4, CCL8, CCL11, CCL20, CXCL1, CXCL2, CXCL3, CXCL10. Interleukins characterized include IL6, IL8, IL10, IL17, and IL23. Immune effectors characterized include GM-CSF, INFγ, CD16, Granzyme B, CD122, and TGFB-1. Markers of defensive immune function characterized include FoxP3, CTLA4, CD25, and GARP-1. Other offensive or defensive immune markers, as well as other chemokines, interleukins or effectors (among other immune-associated markers) can be tested in certain embodiments.

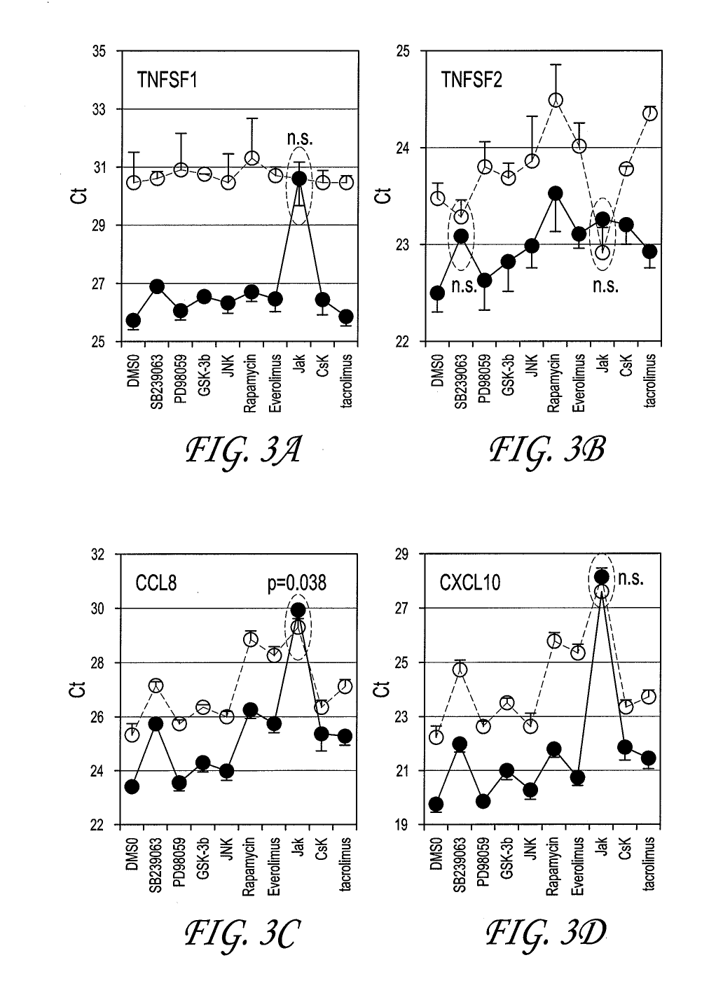

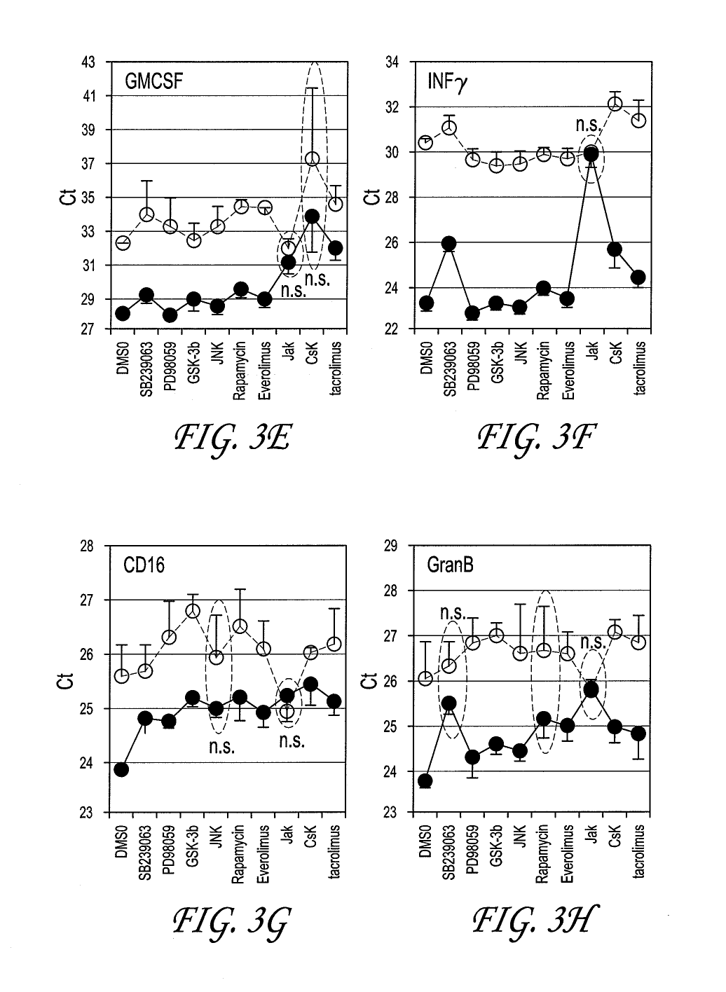

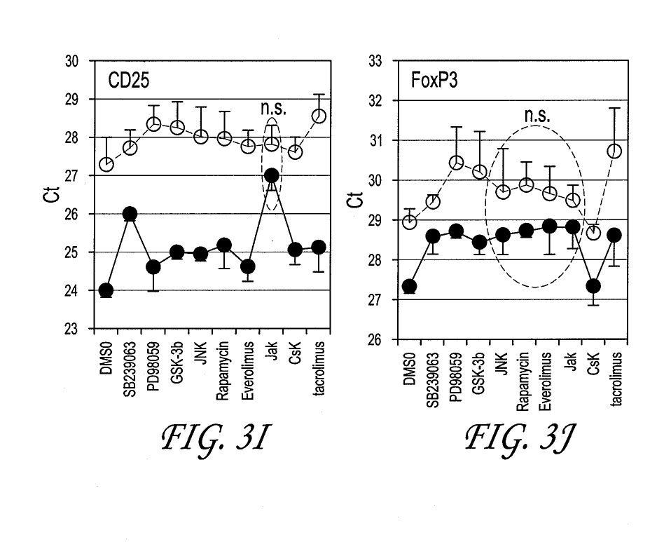

mRNA expression data of offensive and defensive immune markers is show in after rIl-2 stimulation is shown in Table 4. Shaded boxes represent a fold increase of greater than 2 as well as a statistical p value of <0.05. Shown in FIGS. 3A-3J are the expression profiles of selected offensive and defensive markers of immune function and their mRNA expression response to stimulation by PBS (open circles) or 100 ng/mL rIL-2 (closed circles). The various inhibitor compounds used to pre-treat the whole blood samples are listing on the x-axis. As can be seen from this data, the induction of certain offensive or defensive immune markers in the presence of an inhibitor suggest the potential of that compound to promote either offensive or defensive immunity. For example, as shown in FIG. 3A, PBS-stimulation of blood samples and the measurement of induction of TNFSF-1 resulted in no significant expression changes (open circles). However, in the presence of rIL-2 as the stimulant, most blood samples show induction of TNFSF1 mRNA (offensive immune marker), except for the samples treated with an inhibitor of Janus kinase (Jak). This sample showed no induction of TNFSF1 in response to rIL-2. Thus, this compound has a potential to block or reduce the offensive immune response and may function as a pro-immunosuppressant compound. However, when evaluating the effect of the same compound on two markers of the defensive immune response, CD25 or FoxP3 (FIGS. 3I and 3J, respectively), the data indicate that rIL-2 blocks induction of these defensive markers. Thus, from this set of experiments, it is unclear if this particular Jak inhibitor is well-suited for promoting offensive or defensive immune responses. Further dose-response studies and/or studies with additional immune markers are necessary to elucidate the efficacy of this compound.

TABLE 4

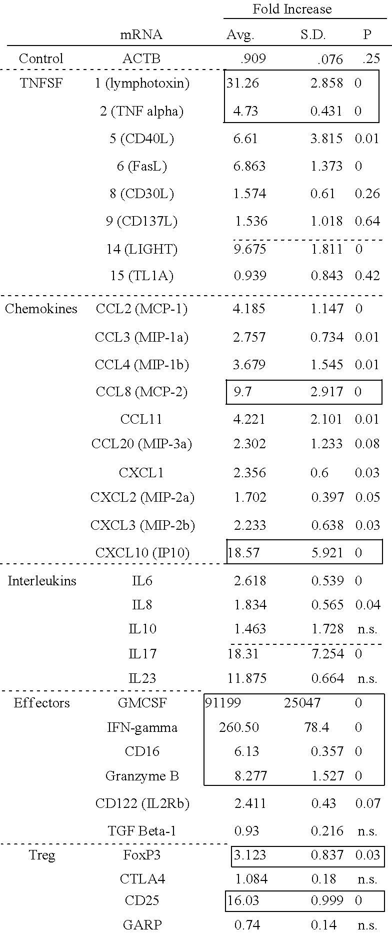

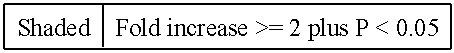

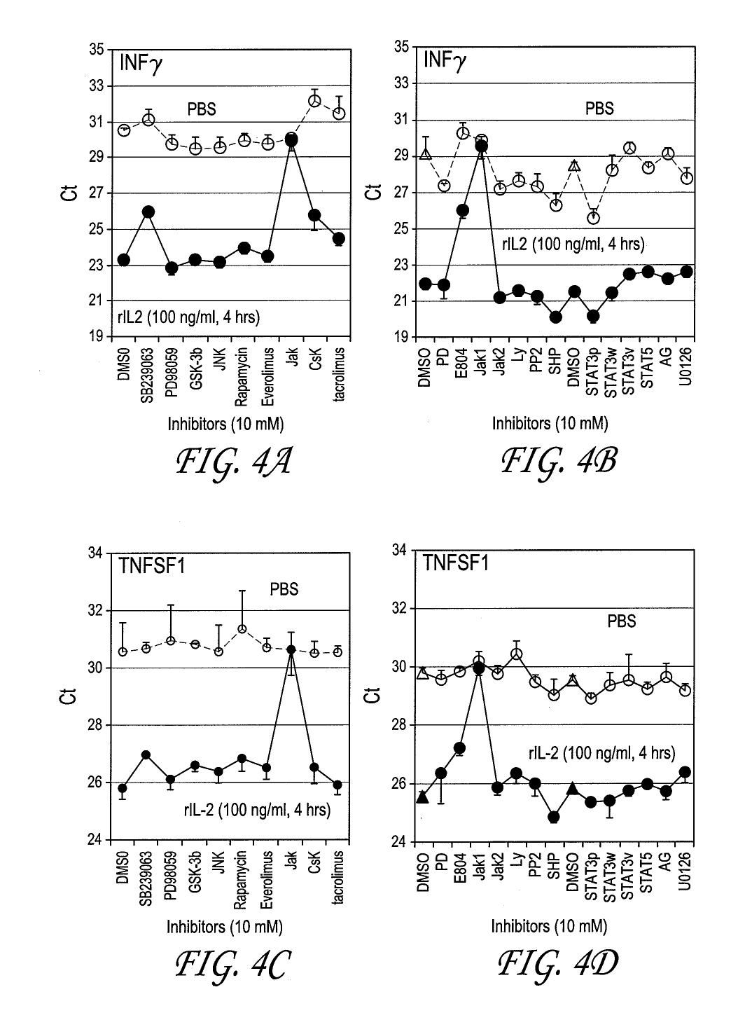

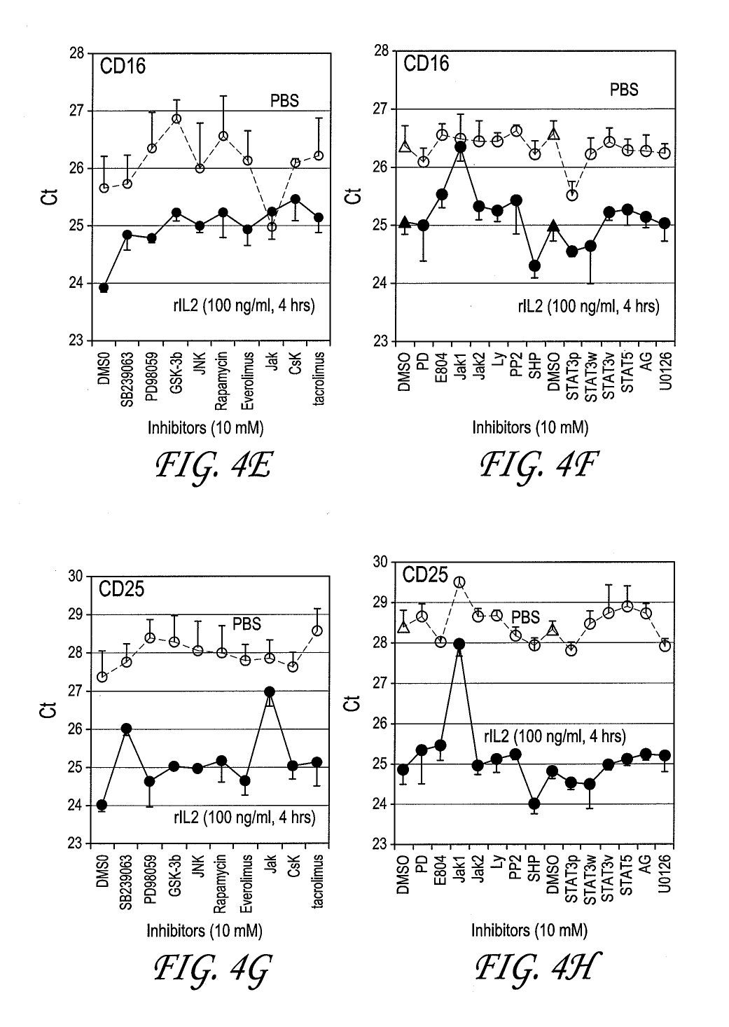

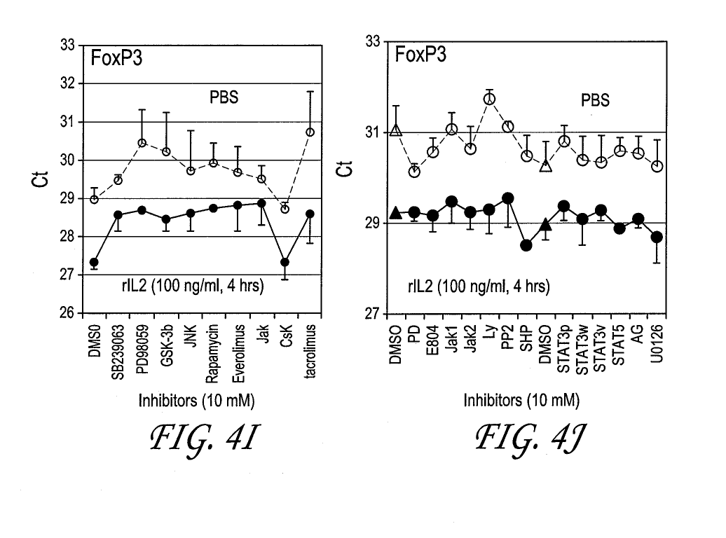

FIGS. 4A-4J depicts additional experiments measuring the induction of IFNγ (FIG. 4A-4B), TNFSF1 (FIG. 4C-4D), CD16 (FIG. 4E-4F), CD25 (FIG. 4G-4H), or FoxP3 (FIG. 4I-4J) in response to PBS or rIL-2 stimulation in the presence or absence of various inhibitor compounds. These data indicate that Jak1 is a specific inhibitor of offensive immune markers (i.e. pro-defensive) as shown by the lack of induction of the offensive markers IFNγ and TNFSF-1 and the induction of expression of FoxP3, a defensive associated marker. Compound E084 also demonstrated a specific inhibition of the offensive markers IFNγ and TNFSF-1. However, E084 induced both CD25 and FoxP3, suggesting that E084 may be a more potent promoter of the defensive immune response. Embodiments of the methods described herein are also well suited to the characterization of various derivative or modified compounds, in that a single assay can generate side by side data comparing of a panel of putative derivative compounds. Moreover, based on the distinct features of the data described above, embodiments of the methods described herein are useful to detecting defensive-specific (i.e. pro-offensive) inhibitor compounds for use in clinical situations. Thus, this assay platform is useful for the analysis/screening of common and selective inhibitors for offensive and defensive immune functions.

### Example 3

### Use of Offensive and Defensive Immune Marker mRNA Induction to Identify Tailored Therapies

Mature leukocytes circulating in peripheral blood are generally in a steady state, and once they migrate to local lesions of inflammation, neoplasms, foreign bodies (microorganisms, transplanted tissues and devices, drugs and vaccines), etc., they are fully activated in a specific manner. The specificity of activation is dependent on, among other factors, the type of leukocyte recruited and the type of local stimulants. In order to simulate in vivo leukocyte responses in an in vitro system, the present example exposed crude whole blood to various specific and general stimulants and the variety of leukocyte responses were quantified. The various leukocyte response were categorized based on their association with a particular type of immune response (e.g., humoral immunity, cell-mediated immunity, etc.)

In most in vitro assays, leukocytes are isolated and cultured with or without specific stimuli for a period of time (e.g., several days to several weeks) to identify functional changes in the leukocytes (e.g., protein synthesis and secretion, apoptosis, cell proliferation, surface marker changes, etc.). However, the complexity, cost, and duration of such assays severely limit their applicability as routine diagnostic tests. Several protocols were developed to attempt to overcome technical difficulties associated with cell isolation and culture conditions, such as, for example use of whole blood with short incubation (typically overnight) with specific stimuli, followed by quantification of adenosine-5′-triphosphate (ATP) levels or measurement of the levels of various cytokines by enzyme-linked immunosorbent assay (ELISA). However, the utility of knowing ATP levels is limited, as a wide variety of leukocyte functions are not ATP-dependent. Further, the detection limit of a typical ELISA is picomole to femtomole (10¹¹ to 10¹⁵ molecules), thus limiting the sensitivity of such protocols.

In contrast, methods according to several embodiments disclosed herein measure the ex vivo induction of leukocyte-function-associated mRNAs. Measuring mRNA levels is advantageous because polymerase chain reaction is capable of sensitivity down to single molecule detection and because mRNA induction happens much earlier than protein synthesis and corresponding biological changes (as would be measured by ELISA). As discussed herein, the use of whole blood maintains the in vivo complex cell-to-cell communication and interaction of leukocytes with plasma factors and proteins. While protocols exist for mRNA analysis in whole blood, such methods provide only a snapshot of gene expression at the time of blood draw. In contrast, embodiments disclosed herein analyze fluctuation in the levels of mRNA after appropriate stimulation, which provides a dynamic series of data points related to the leukocyte responses to stimuli.

Although PCR is sensitive enough to detect a single copy of target gene, several embodiments disclosed herein are particularly sensitive based on the reduced variation among triplicate aliquots of whole blood. In standard assays, variation can be induced at any step from leukocyte isolation, RNA purification, cDNA synthesis, to PCR. Because of the amplification-based nature of PCR, even minute variations introduced before PCR will be exponentially increased. Moreover, even from a single blood sample, many aliquots are generated based on the number of stimulants, dose responses, time course, combinations of stimulants, duplicate or triplicate, etc. Based on such limitations of the standard protocols known in the art, Applicant developed a high throughput assay platform, which is exploited in several embodiments disclosed herein. Further information regarding the assay platform may be found in U.S. patent applications Ser. Nos. 10/796,298, 11/525,515, 11/376,018, 11/803,593, 11/803,594, and 11/803,663, each of which is incorporated in its entirety by reference herein.

### Materials and Methods

### Materials

Anti-T cell receptor α/β chain (TCR) monoclonal antibody (IgG1_{K}) and control mouse IgG1_{K} were obtained from BioLegend (San Diego, Calif., USA). Reverse transcriptase, dNTP, and RNasin were purchased from Invitrogen (Carlsbad, Calif., USA). All other chemicals were purchased from Sigma-Aldrich (St. Louis, Mo., USA). Immune complex (heat aggregated IgG, HAG) was prepared by heating human IgG at 63° C. for 15 min as described previously (Ostreiko et al., 1987). In 8-well strip microtubes, 1.2 μl each of phytohemagglutinin-L (2 mg/ml), HAG (10 mg/ml), lipopolysaccharide (LPS) (0.5 mg/ml), zymosan A (75 mg/ml), recombinant interleukin 2 (rIL2) (5 μg/ml), phosphate buffered saline (PBS), anti-TCR antibody (50 μg/ml), and control IgG (50 μg/ml), were added respectively into 8 wells, and stored at −80° C. until use.

### Blood Treatment

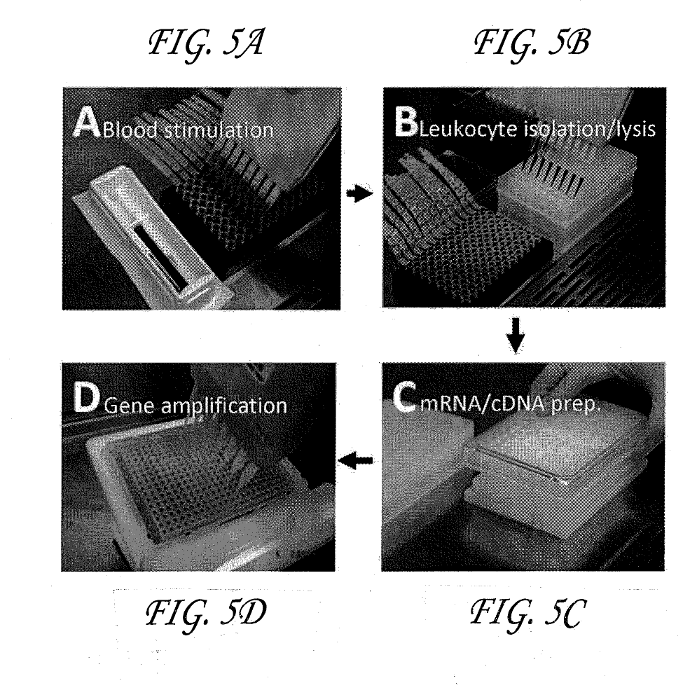

Heparinized whole blood samples were obtained from Apex Research Institute (Tustin, Calif., USA) after Institutional Review Board approvals. In order to equalize post-blood collection condition, blood samples were stored at 4° C. overnight. Next morning, blood was decanted into a reservoir and using an 8-well multi-channel pipette, 60 μl each of blood was dispensed into 3 strips containing the control agents or stimulants described above (FIG. 5A). The blood volume needed for this test was 1.44 ml (60 μl/well×8 wells×3 strips (triplicate)). After cap was closed, strips were incubated at 37° C. for 4 hours, then stored frozen at −80° C. It shall be appreciated that, in some embodiments, larger or smaller volumes of blood may be used (e.g., increased due to testing a larger number of stimulants or leukocyte-activation related genes). Two categories of patient were tested in this example, a normal healthy (control) subject (data in Table 5) and a patient having cancer (data in Table 6).

Target mRNAs

The mRNA sequences of target mRNAs were retrieved from GenBank. PCR primers were designed within the coding region by Primer Express (Applied Biosystems (ABI), Foster City, Calif., USA) (See Table 1). Oligonucleotides were synthesized by IDT (Coralville, Iowa, USA). The target mRNAs (total 32) were β-actin (ACTB), β2-microglobulin (B2M), granzyme B (GZB), perforin 1 (PRF1), tumor necrosis factor superfamily (TNFSF)-1, 2, 5, 14, and 15, CCL chemokines-2, 4, 8, and 20, CXCL chemokines-3, and 10, interleukin (IL)-2, 4, 6, 8, 10, and 17A, interferon-γ (IFNG), granulocyte-macrophage colony-stimulating factor (GMCSF), CD11a, 16 and 25, transforming growth factor beta 1 (TGFB1), forkhead box P3 (FOXP3), immunoglobulin heavy locus (IGH@), arginase (ARG), vascular endothelial growth factor (VEGF), and pro-opiomelanocortin (POMC).

mRNA Analysis

Fifty microliters of stimulated and frozen whole blood was thawed and applied to 96-well custom filterplates (FIG. 5B). Leukocytes were isolated on the filter membranes by centrifugation. In some embodiments, other techniques may be used to isolate the leukocytes on the filter membrane (e.g., vacuum, positive pressure, gravity and the like). Further information on isolation of leukocytes may be found in U.S. patent application Ser. Nos. 10/796,298, 11/525,515, 11/376,018, 11/803,593, 11/803,594, and 11/803,663, each of which is incorporated in its entirety by reference herein. Sixty μL of lysis buffer containing a cocktail of specific reverse primers was applied to the filterplates, and the resultant cell lysates were transferred to oligo(dT)-immobilized microplates for poly(A)+ mRNA purification (FIG. 5C). The cDNA was directly synthesized in 50 μL solutions at each well: specific primer-primed cDNA in the liquid phase and oligo(dT)-primed cDNA in the solid phase. The liquid and solid phase cDNAs were used for real time PCR using iTaqSYBR (Biorad, Hercules, Calif., USA) in thermal cyclers (PRISM 7900, ABI, and iCycler, Biorad, respectively). PCR conditions were 95° C. for 10 min followed by 50 cycles of 65° C. for 1 min and 95° C. for 30 sec. For IL2 and IL4, 4 μl of undiluted cDNA solution was used in the final volume of 10 μl in 384 well PCR plate. (FIG. 5D). For FOXP3, ARG, IFNG, GMCSF, and POMC, 2 μl of undiluted cDNA solution was used for PCR in the final volume of 5 μL. The cDNA was then diluted 1:2 by adding 32 μL of DNase/RNase free water, and 2 μl each of cDNA was used for PCR for remaining 24 genes (except IL17A) in the final volume of 5 μL. After the leftover cDNA was transferred to fresh strip microtubes, solid phase cDNA was used directly to amplify IL17A by adding 30 μL PCR solution. The melting curve was always analyzed to confirm that PCR signals were derived from a single PCR product. The cycle threshold (Ct) was determined by analytical software (SDS, ABI), and statistical p values were calculated by t-test using 3 Ct values each of stimulant and control. The Ct of drug-treated triplicate samples were subtracted individually by the mean Ct values of control samples to calculate ΔCt, and the fold increase was calculated as 2̂(−ΔCt).

### Results and Discussion

In the present example, 6 different stimulants (with 2 controls) were used (Tables 5 and 6, x-axis). PHA is a lectin commonly used to stimulate the general T-cell population. HAG is a model of immune complex to stimulate IgG Fc receptor (FcγR)-positive leukocytes. Unlike antibody-dependent cell-mediated cytotoxicity (ADCC), where a primary cellular target is CD16+ natural killer (NK) cells, stimulation with HAG targets CD16+, CD32+, and CD64+ cells. LPS and zymosan are commonly used agents to stimulate toll-like receptor (TLR) for the analysis of innate immunity. Since the anti-TCR antibody binds to the antigen recognition molecule of the T-cell receptor (α/β chain) of both CD4+ and CD8+ T-cells, it is used as a universal TCR antigen (Mitsuhashi et al., 2008c). Recombinant IL-2 binds to IL2-receptor positive T-cells, including regulatory T-cells (Treg), and induces a wide variety of immunological reactions.

Although immunity is extremely complex with numerous cellular and humoral factors interacting by each other, for purposes of predicting an effective individualized therapy as in several embodiments disclosed herein, such complexity can be simplified down to several functional categories as shown on in Tables 5 and 6.

Generally, in order for leukocytes to relocate from blood to local lesions, they first express CD11a on the cell surface, which binds to intercellular adhesion molecules expressed on endothelial cells damaged by a lesion. As shown in Tables 5 and 6, detection of increased CD11a expression was detected by stimulation of whole blood samples with zymosan. Once CD11a-expressing leukocytes encounter target cells or molecules (e.g., those of the lesion), appropriate subsets of leukocytes must be recruited to the lesion. Expression of a variety of chemotactic CCL and CXCL chemokines were analyzed. CCL2 (monocytes and basophils), CCL4 (granulocytes), CCL8 (mast cells, eosinophils, basophils, monocytes, T cells, and NK cells), CCL20 (lymphocytes), CXCL3 (monocytes), CXCL8 (=IL8) (neutrophils), and CXCL10 (monocytes, macrophages, T cells, NK cells, and dendritic cells) are known as chemoattractants for respectively listed cell types. As shown in Tables 5 and 6, each stimulant induced different subsets of chemokines, which indicates a possible means to exploit (e.g., recruit) particular cell types based on an individual's chemotactic and chemokine expression profile. In some embodiments, an individual's chemotactic and chemokine expression profile provides data that enables a prediction of that individual's ability to develop cellular infiltration (e.g., deliver the appropriate immune cells to the target site) at disease sites. Such data is useful, in some embodiments, to predict the efficacy of and develop a tailored therapy. For example, if stimulation of whole blood of a cancer patient fails to induce expression of any chemotactic or chemokine molecules, that individual may be deficient in leukocyte recruiter function, and therefore would not be an ideal candidate for an immune based therapy (e.g., cancer immunotherapy). Rather, that individual is likely to respond better to a more traditional radiation, pharmacological, or surgical approach.

Once recruited to the site of a lesion, locally infiltrated leukocytes also induce a cascade of events designed to kill the target cells. Multiple mechanisms for killing a target cell can be employed, including induction of apoptosis. As shown in Tables 5 and 6, induction of Granzyme and perforin mRNA (well known inducers of apoptosis) by various stimulants were identified. Increased expression of CD16, a specific marker of NK cells was also identified. The Tumor Necrosis Factor (ligand) Super Family (TNFSF) comprises a variety of inducers of apoptosis against target cells that are TNFSF receptor-positive. As shown in Tables 5 and 6, each stimulant induced different members of TNFSF. Thus, the data related to the offensive immune function of an individual may be used, in some embodiments, to characterize the potential of that patient to generate innate anti-cancer activity. Also, in some embodiments, the severity of autoimmune diseases can be characterized. Advantageously, the methods presented herein allow a characterization of the potential for a patient to respond to a particular type of therapy prior to administering the therapy. This is particularly advantageous in situations where a patient's survival is dependent on initiating an effective therapy as early as possible. As such, the knowledge that a cancer patient exhibits little inducement of expression of “killer” molecules suggests that such a patient would likely not benefit from such therapies and that other, non-immune, therapies should be investigated.

As discussed above, human immunity has a variety of negative regulators (also referred to herein as “defensive immune markers”), which include humoral (IL10 and TGFB1), and cellular components (Treg and myeloid derived suppressor cells (MDSC)). These regulators have the capacity to suppress or reduce the efficacy of an offensive (e.g., killer) immune response when induced. In contrast, their reduced expression may favor generation of auto-immune disorders. Thus, the balance of expression of negative regulators and recruiter/killer molecules are used, in some embodiments, to further assess the potential efficacy of a given type of therapy. As shown in Table 5, IL10 mRNA was induced by the anti-TCR antibody and zymosan, and TGFB1 mRNA was induced by zymosan. FOXP3 and CD25 mRNA were measured as the markers of Treg activity. While no stimulant induced FoxP3, CD25 was induced by all stimulants except HAG (see Table 5). Arginase mRNA was measured as a marker of MSDC function, though no induction was detected in Table 5.

As shown in Table 6, a patient with cancer appeared to have a greater degree of suppressor induction in response to the various stimuli. Zymosan significantly induced all suppressors analyzed except for TGF-beta1. Similarly, PHA induced 3 of the 5 suppressor markers analyzed. Thus in some embodiments, such data may suggest the origins of an illness, for example the enhanced expression of suppressors may have contributed to a reduced efficacy of killer (e.g., offensive) function, thereby facilitating the generation of a malignancy. Likewise, enhanced suppressor function, particularly in the absence of enhanced killer function, suggests that immune therapies would be ineffective for such an individual.

As the markers of various subsets of T helper cells, which primarily function to activate and direct other types of immune cells, the induction of IL2 (Th1), IFNG (Th1), IL4 (Th2), IL10 (Th2), and IL17 (Th17) mRNAs were identified (Tables 5 and 6). Induction of these markers suggest that there is capacity for multiple levels of the immune system are upregulated simultaneously, rather than simply an induction of offensive (e.g., killer) function cells. As such, in some embodiments, analysis of each of the various functional categories discussed above, taken together, are used to determine which type of treatment modality is likely to be effective. For example, upregulation of offensive markers in conjunction with a lack of induction of helper or recruitment markers suggests that the offensive cells, while having greater activity, may not be sufficiently well targeted or supported by other portions of the immune system. In such a case, immune-based therapies may not be ideal. In other embodiments, however, the magnitude of induction of a single category (or single marker) may be sufficient to merit an associated therapeutic regimen.

A variety of other functions are carried out in the immune system, such as the ability of the immune system to “learn” the identity of various foreign molecules, which is of critical importance in the humoral (e.g., antibody mediated) immune response. Granulocyte macrophage colony stimulating factor (GM-CSF) is functions as a white blood cell growth factor, and GMCSF mRNA was modestly induced by various stimulations in a healthy subject (Table 5). Likewise, GMCSF was upregulated in a cancer patient, though to a vastly greater degree than the healthy subject (Table 6). These data suggest that a cancer patient may have the capacity to make a large number of leukocytes, but the total number of cells may not guarantee effective immune function, but rather the eventual balance of the offensive and defensive functions may be a significant determining factor. As a marker of B-cells, which function to make antibodies against antigens and develop into memory B-cells, mRNA of IgG heavy chain (IGH@) was induced by zymosan in a healthy patient (Table 5), but not in a cancer patient (Table 6). Many lesions, such as cancers require an increased blood supply due to the higher rate of cellular metabolism and tissue growth. VEGF expression was studied as a marker of angiogenesis and was induced by zymosan in both the healthy and cancer patient. Although pain-associated POMC (endorphin) mRNA was not induced in these subjects, POMC mRNA has been shown to be induced by zymosan in other healthy control subjects in related experiments (data not shown).

As demonstrated in this example, the high-throughput methods were sensitive enough to characterize a wide spectrum of leukocyte function in a healthy individual and in an individual (Table 5) with cancer (Table 6). Using triplicate aliquots of whole blood for both stimulant and solvent controls, statistical conclusion were able to be drawn for each stimulant for each mRNA. While control genes such as ACTB and B2M were not induced (e.g., fold change >2) in the control individual, B2M was induced in the cancerous individual. However, since B2M is a component of the major histocompatibility complex, it is not unreasonable to consider that alterations in expression would be induced by stimulation of whole blood with zymosan. Regardless, control genes were not used to normalize the PCR results of other mRNAs. In fact, given the sensitivity of the methods used herein, statistical significance was often identified even fold increase was <2, or >0.6 (e.g., rIL2- and PHA-induced ACTB, PHA-induced CCL4, zymosan-induced CXCL10, HAG- and zymosan-induced FOXP3 in Table 5). In contrast, some low copy number genes (e.g., IL2, IL4, GMCSF, and POMC) exhibit large variation, and occasionally >10 fold increase was not significant (TCR-induced GMCSF in Table 5). Thus in Tables 5 and 6, >2 fold increase plus p<0.05 was considered as positive results (e.g., significant induction; dark background). The degree of induction of the various markers may not necessarily be directly linked to the degree of biological significance, for example, a large fold increase in expression of a low copy number gene may have less biological impact as compared to a small fold increase of an abundant gene. Thus, while the degree of change in expression may not necessarily be indicative of the degree of change in a particular pathway, these data are of particular value for determining the patterns of expression. As disclosed herein, the pattern of expression, in several embodiments, is used to make develop an individualize therapy or diagnosis based on the expression of certain categories of leukocyte-function associated markers. With the inclusion of general immune function stimulators such as rIL2, data generated by the methods disclosed herein are applicable to oncology and autoimmune diseases, among other disease types. According to several embodiments, several embodiments use the data generated in relation to developing therapies in the context of preclinical studies, clinical trials, as well as the development of companion diagnostics.

TABLE 5
Leukocyte Function Profile of Control Patient

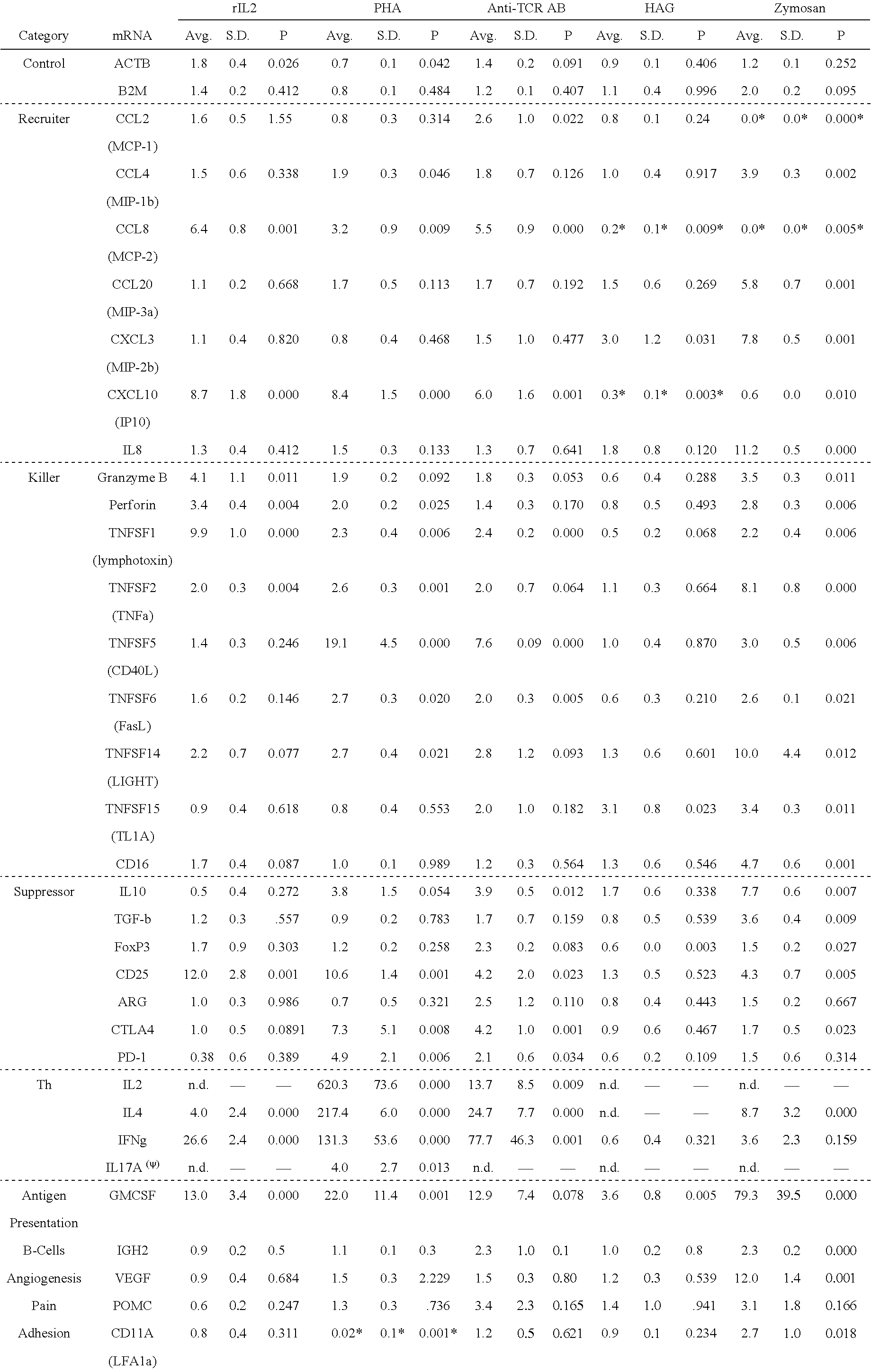

^{ψ} IL17A was amplified directly on the oligo(dT) plate
n.d. = not detected

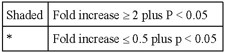

TABLE 6
Leukocyte Function Profile of Cancer Patient

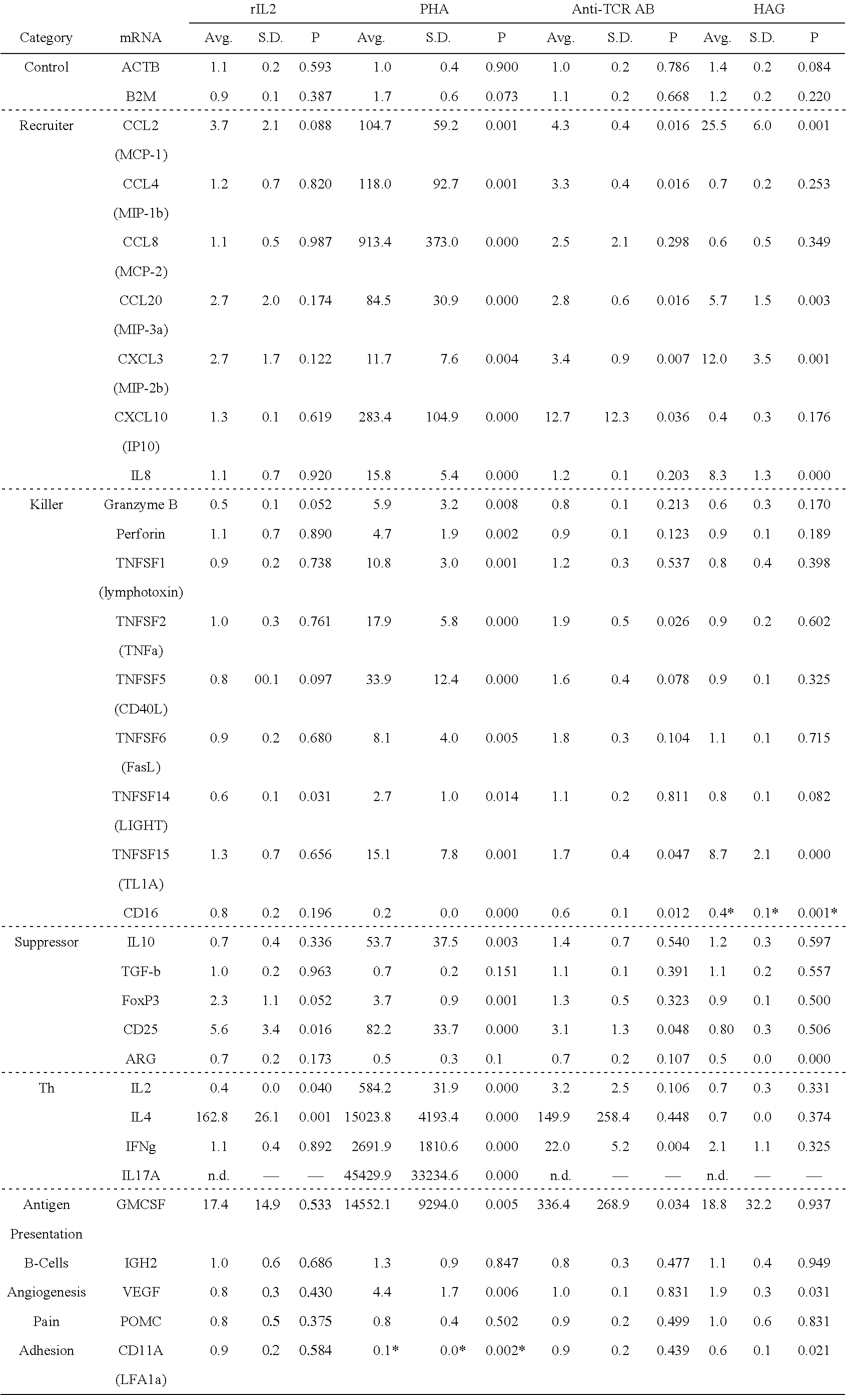

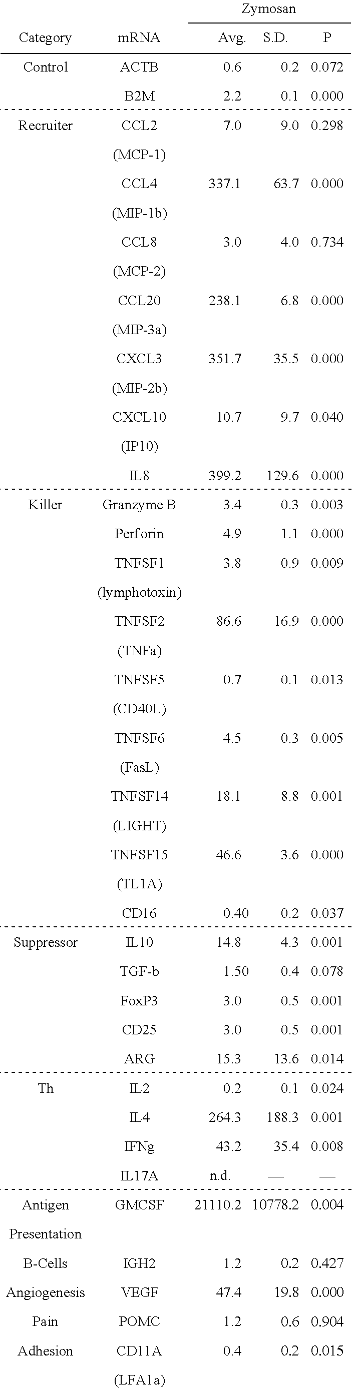

n.d. = not detected

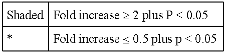

1.-34. (canceled)

35. A method for enabling a medical professional to recommend an immune-based or non-immune-based therapy to a subject, the method comprising:
obtaining at least a first and a second aliquot of whole blood from the subject;
exposing the first aliquot to a solvent and exposing the second aliquot to the solvent further comprising an immune cell stimulating agent;
quantifying an amount of mRNA encoding an immune cell function-related mRNA in each of the first and second aliquots;
calculating a ratio of the amount the immune cell function-related mRNA in the first aliquot to the amount of immune cell function-related mRNA in the second aliquot; and
1) indicating to a medical professional whether the ratio is less than 1 or greater than 1 so as to enable the medical professional to recommend an immune-based therapy if the ratio is less than 1 and a non-immune-based therapy if the ratio is greater than 1,
a) wherein the immune stimulating agent is zymosan A and the immune cell function-related mRNA is IGH2, or
b) wherein the immune stimulating agent is zymosan A and the immune cell function-related mRNA is CD16; or
2) indicating to a medical professional whether the ratio is less than 1 or greater than 1 so as to enable the medical professional to recommend an non-immune-based therapy if the ratio is less than 1 and an immune-based therapy if the ratio is greater than 1,
a) wherein the immune stimulating agent is recombinant interleukin-2 (rIL2) and the immune cell function-related mRNA is FOXP3, or
b) wherein the immune stimulating agent is zymosan A and the immune cell function-related mRNA is arginase 1.

36. The method of claim 35, wherein said whole blood is stored at room temperature or refrigeration for less than 1 day.

37. The method according to claim 35, wherein the exposing of said first and said second aliquots is for less than 24 hours.

38. The method of claim 37, wherein said exposing is for between 2 and 6 hours.

39. The method of claim 38, wherein said exposing is for about 4 hours.

40. The method of claim 35, wherein said whole blood is optionally heparinized.

41. The method of claim 35, wherein the subject is in need of therapy for cancer.

42. The method of claim 35, wherein the immune-based therapy comprises an anti-cancer vaccine.

43. The method of claim 35, wherein the immune-based therapy comprises an anti-cancer vaccine.

44. The method of claim 35, wherein the non-immune-based therapy comprises one or more of radiation, pharmacological, and surgical approaches.

45. The method of claim 35, wherein said medical professional administers the recommended immune-based or non-immune-based therapy.

46. A method for advising a therapy to a subject based on the subject's immune cell function function, the method comprising:
ordering a test of the subject's blood, said test comprising:
obtaining at least a first and a second aliquot of heparinized whole blood from the subject;
exposing the first aliquot to a solvent and exposing the second aliquot to the solvent further comprising an immune cell stimulating agent;
quantifying an amount of mRNA encoding an immune cell function-related mRNA in each of the first and second aliquots;
calculating a ratio of the amount the immune cell function-related mRNA in the first aliquot to the amount of immune cell function-related mRNA in the second aliquot; and
1) advising the subject to undergo an immune-based therapy if the ratio is less than 1 and a non-immune-based therapy if the ratio is greater than 1,
a) wherein the immune stimulating agent is zymosan A and the immune cell function-related mRNA is IGH2, or
b) wherein the immune stimulating agent is zymosan A and the immune cell function-related mRNA is CD16; or
2) advising the subject to undergo a non-immune-based therapy if the ratio is less than 1 and an immune-based therapy if the ratio is greater than 1,
a) wherein the immune stimulating agent is recombinant interleukin-2 (rIL2) and the immune cell function-related mRNA is FOXP3, or
b) wherein the immune stimulating agent is zymosan A and the immune cell function-related mRNA is arginase 1.

47. The method of claim 46, further comprising administering to said subject a non-immune-based therapy.

48. The method of claim 46, wherein said whole blood is stored at room temperature or refrigeration for less than 1 day.

49. The method according to claim 46, wherein the exposing of said first and said second aliquots is for less than 24 hours.

50. The method of claim 49, wherein said exposure is for about 4 hours.

51. The method of claim 46, wherein said whole blood is optionally heparinized.

52. The method of claim 46, wherein the immune-based therapy comprises an anti-cancer vaccine and wherein the non-immune-based therapy comprises one or more of radiation, pharmacological, and surgical approaches.

53. The method of claim 35, wherein said medical professional administers the recommended immune-based or non-immune-based therapy.

54. A method for treating a subject having cancer based on the subject's regulatory immune cell function, the method comprising:
ordering a test of the subject's blood, said test comprising:
obtaining at least a first and a second aliquot of heparinized whole blood from the subject;
exposing the first aliquot to a solvent and exposing the second aliquot to the solvent further comprising an immune cell stimulating agent;
quantifying an amount of mRNA encoding an immune cell function-related mRNA in each of the first and second aliquots;
calculating a ratio of the amount the immune cell function-related mRNA in the first aliquot to the amount of immune cell function-related mRNA in the second aliquot; and
1) administering to the an immune-based therapy if the ratio is less than 1 and a non-immune-based therapy if the ratio is greater than 1,
a) wherein the immune stimulating agent is zymosan A and the immune cell function-related mRNA is IGH2, or
b) wherein the immune stimulating agent is zymosan A and the immune cell function-related mRNA is CD16; or
2) administering to the subject a non-immune-based therapy if the ratio is less than 1 and an immune-based therapy if the ratio is greater than 1,
a) wherein the immune stimulating agent is recombinant interleukin-2 (rIL2) and the immune cell function-related mRNA is FOXP3, or
b) wherein the immune stimulating agent is zymosan A and the immune cell function-related mRNA is arginase 1.


SEQUENCE LISTING (72 sequences)

SEQ ID NO: 1 - DNA, 18 bp, Artificial Sequence
  Synthesized PCR primer
cctggcacccagcacaat

SEQ ID NO: 2 - DNA, 20 bp, Artificial Sequence
  Synthesized PCR primer
tcatgaggcacacctagccg

SEQ ID NO: 3 - DNA, 23 bp, Artificial Sequence
  Synthesized PCR primer
tgactttgtcacagcccaagata

SEQ ID NO: 4 - DNA, 20 bp, Artificial Sequence
  Synthesized PCR primer
tccaaacttctacggcgtaa

SEQ ID NO: 5 - DNA, 20 bp, Artificial Sequence
  Synthesized PCR primer
ccattgtggccaaggagatc

SEQ ID NO: 6 - DNA, 19 bp, Artificial Sequence
  Synthesized PCR primer
aggtacctggtggacctgt

SEQ ID NO: 7 - DNA, 23 bp, Artificial Sequence
  Synthesized PCR primer
ggtattccaaaccaaaagaagca

SEQ ID NO: 8 - DNA, 26 bp, Artificial Sequence
  Synthesized PCR primer
tcatgcacatactggaccttgacttg

SEQ ID NO: 9 - DNA, 26 bp, Artificial Sequence
  Synthesized PCR primer
agagctacacaagaatcaccaacatc

SEQ ID NO: 10 - DNA, 19 bp, Artificial Sequence
  Synthesized PCR primer
tttgccccgttcctccaga

SEQ ID NO: 11 - DNA, 28 bp, Artificial Sequence
  Synthesized PCR primer
gatacacagaccgtattcttcatcctaa

SEQ ID NO: 12 - DNA, 26 bp, Artificial Sequence
  Synthesized PCR primer
acactgtagttacgatagtagaaagt

SEQ ID NO: 13 - DNA, 23 bp, Artificial Sequence
  Synthesized PCR primer
ggaattcacctcaagaacatcca

SEQ ID NO: 14 - DNA, 21 bp, Artificial Sequence
  Synthesized PCR primer
ggtttggcttcagtatcggtg

SEQ ID NO: 15 - DNA, 21 bp, Artificial Sequence
  Synthesized PCR primer
tccacgtgttgagatcattgc

SEQ ID NO: 16 - DNA, 21 bp, Artificial Sequence
  Synthesized PCR primer
gtcttagcttccggtagttct

SEQ ID NO: 17 - DNA, 27 bp, Artificial Sequence
  Synthesized PCR primer
tgctaaagaacttagatgtcagtgcat

SEQ ID NO: 18 - DNA, 23 bp, Artificial Sequence
  Synthesized PCR primer
actctcactaactctcacctggt

SEQ ID NO: 19 - DNA, 20 bp, Artificial Sequence
  Synthesized PCR primer
gcggtggcttcctgatacaa

SEQ ID NO: 20 - DNA, 23 bp, Artificial Sequence
  Synthesized PCR primer
ttcgaggtatttacagtggaacc

SEQ ID NO: 21 - DNA, 20 bp, Artificial Sequence
  Synthesized PCR primer
tccttggcacctgtgatcag

SEQ ID NO: 22 - DNA, 22 bp, Artificial Sequence
  Synthesized PCR primer
actctacgttggacttagtacc

SEQ ID NO: 23 - DNA, 22 bp, Artificial Sequence
  Synthesized PCR primer
cagctatccacccacacagatg

SEQ ID NO: 24 - DNA, 22 bp, Artificial Sequence
  Synthesized PCR primer
tgacagaagaaacctcggaagc

SEQ ID NO: 25 - DNA, 22 bp, Artificial Sequence
  Synthesized PCR primer
cgaaggctccaaagaagacagt

SEQ ID NO: 26 - DNA, 20 bp, Artificial Sequence
  Synthesized PCR primer
tgaaaccctagtaacgggac

SEQ ID NO: 27 - DNA, 19 bp, Artificial Sequence
  Synthesized PCR primer
ccacagttccgccaaacct

SEQ ID NO: 28 - DNA, 24 bp, Artificial Sequence
  Synthesized PCR primer
ctcataaacttaacgttggtccac

SEQ ID NO: 29 - DNA, 24 bp, Artificial Sequence
  Synthesized PCR primer
tggcagcatcttcacttctaaatg

SEQ ID NO: 30 - DNA, 24 bp, Artificial Sequence
  Synthesized PCR primer
tctctacaaaacccctgagtaaag

SEQ ID NO: 31 - DNA, 18 bp, Artificial Sequence
  Synthesized PCR primer
cgtccgtgtgctggatga

SEQ ID NO: 32 - DNA, 22 bp, Artificial Sequence
  Synthesized PCR primer
cagaatgaagccccgaaagtac

SEQ ID NO: 33 - DNA, 23 bp, Artificial Sequence
  Synthesized PCR primer
tgcgaagtaggtagcaactggtt

SEQ ID NO: 34 - DNA, 22 bp, Artificial Sequence
  Synthesized PCR primer
gttcttcccctgttcgattacc

SEQ ID NO: 35 - DNA, 22 bp, Artificial Sequence
  Synthesized PCR primer
gtttggcagtgtcaaccatctc

SEQ ID NO: 36 - DNA, 23 bp, Artificial Sequence
  Synthesized PCR primer
acgaaccactaccatgaggaaaa

SEQ ID NO: 37 - DNA, 24 bp, Artificial Sequence
  Synthesized PCR primer
gccatgagtgagtttgacatcttc

SEQ ID NO: 38 - DNA, 29 bp, Artificial Sequence
  Synthesized PCR primer
atcctgtatttaatctccagaggttttag

SEQ ID NO: 39 - DNA, 24 bp, Artificial Sequence
  Synthesized PCR primer
ctgctgaggctcaagttaaaagtg

SEQ ID NO: 40 - DNA, 21 bp, Artificial Sequence
  Synthesized PCR primer
tgttaaggaccgctatggagt

SEQ ID NO: 41 - DNA, 19 bp, Artificial Sequence
  Synthesized PCR primer
cacctacgccacgctcatc

SEQ ID NO: 42 - DNA, 20 bp, Artificial Sequence
  Synthesized PCR primer
aagtgtgcgtacaaacggaa

SEQ ID NO: 43 - DNA, 22 bp, Artificial Sequence
  Synthesized PCR primer
cagaagtcatgaagcccaagtg

SEQ ID NO: 44 - DNA, 20 bp, Artificial Sequence
  Synthesized PCR primer
tctgtaggcaacacgaacgg

SEQ ID NO: 45 - DNA, 22 bp, Artificial Sequence
  Synthesized PCR primer
catgcctcctcttcttccttga

SEQ ID NO: 46 - DNA, 20 bp, Artificial Sequence
  Synthesized PCR primer
atcagtaccaccgtgggagg

SEQ ID NO: 47 - DNA, 19 bp, Artificial Sequence
  Synthesized PCR primer
ctcagccgtgcctgtgttc

SEQ ID NO: 48 - DNA, 23 bp, Artificial Sequence
  Synthesized PCR primer
ctcatacggtggtaacagaaagg

SEQ ID NO: 49 - DNA, 26 bp, Artificial Sequence
  Synthesized PCR primer
agacaccagaagaagtaactcgaaca

SEQ ID NO: 50 - DNA, 19 bp, Artificial Sequence
  Synthesized PCR primer
caaagcctgaacgagccct

SEQ ID NO: 51 - DNA, 27 bp, Artificial Sequence
  Synthesized PCR primer
gaactaaagggatctgaaacaacattc

SEQ ID NO: 52 - DNA, 24 bp, Artificial Sequence
  Synthesized PCR primer
aaaacagtttcgtagtagagttgt

SEQ ID NO: 53 - DNA, 20 bp, Artificial Sequence
  Synthesized PCR primer
cacaggcacaagcagctgat

SEQ ID NO: 54 - DNA, 23 bp, Artificial Sequence
  Synthesized PCR primer
gaacttaaggacaggacacttcc

SEQ ID NO: 55 - DNA, 23 bp, Artificial Sequence
  Synthesized PCR primer
ggagaccatcaaggaagacatga

SEQ ID NO: 56 - DNA, 20 bp, Artificial Sequence
  Synthesized PCR primer
aacttacaggttgcgtttcg

SEQ ID NO: 57 - DNA, 21 bp, Artificial Sequence
  Synthesized PCR primer
gaaatccaggatgcccaaatt

SEQ ID NO: 58 - DNA, 22 bp, Artificial Sequence
  Synthesized PCR primer
agttggacttgtaggtattggc

SEQ ID NO: 59 - DNA, 18 bp, Artificial Sequence
  Synthesized PCR primer
ggccccttgaccatgatg

SEQ ID NO: 60 - DNA, 21 bp, Artificial Sequence
  Synthesized PCR primer
tttgaaggacacgttgggtct

SEQ ID NO: 61 - DNA, 23 bp, Artificial Sequence
  Synthesized PCR primer
cagccggagaacaactacaagac

SEQ ID NO: 62 - DNA, 19 bp, Artificial Sequence
  Synthesized PCR primer
ctgttctcgtccaccgtcg

SEQ ID NO: 63 - DNA, 20 bp, Artificial Sequence
  Synthesized PCR primer
cgcagctactgccatccaat

SEQ ID NO: 64 - DNA, 24 bp, Artificial Sequence
  Synthesized PCR primer
ctctagctcatgtagaagttcggt

SEQ ID NO: 65 - DNA, 19 bp, Artificial Sequence
  Synthesized PCR primer
acgagggcccctacaggat

SEQ ID NO: 66 - DNA, 21 bp, Artificial Sequence
  Synthesized PCR primer
acaagtttttgcggtagtagt

SEQ ID NO: 67 - DNA, 19 bp, Artificial Sequence
  Synthesized PCR primer
gacctgatctgccgcttca

SEQ ID NO: 68 - DNA, 19 bp, Artificial Sequence
  Synthesized PCR primer
taggaggagtggtgcgacc

SEQ ID NO: 69 - DNA, 22 bp, Artificial Sequence
  Synthesized PCR primer
ggagatcctcgtccaagtgatc

SEQ ID NO: 70 - DNA, 20 bp, Artificial Sequence
  Synthesized PCR primer
gagataccgtcgttgcggag

SEQ ID NO: 71 - DNA, 28 bp, Artificial Sequence
  Synthesized PCR primer
catatttacaacagagtaccaggtagca

SEQ ID NO: 72 - DNA, 26 bp, Artificial Sequence
  Synthesized PCR primer
ggttttcttgttcttaaagaaccatt